(19) United States
(12) Patent Application Publication
(10) Pub. No.: US 2025/0222121 A1
(43) Pub. Date: Jul. 10, 2025

(54) MULTIVALENT SMAC/DIABLO PROTEIN PEPTIDOMIMETIC COVALENTLY LINKED TO A TUMOR-HOMING PEPTIDE FOR TREATING CANCERS

(71) Applicant: BIOXEL INC., Cambridge, MA (US)

(72) Inventors: Patrick R. GLEASON, Millis, MA (US); Elazer R. EDELMAN, Brookline, MA (US)

(73) Assignee: BIOXEL INC., Cambridge, MA (US)

(21) Appl. No.: 18/917,887

(22) Filed: Oct. 16, 2024

Related U.S. Application Data
(60) Provisional application No. 63/590,960, filed on Oct. 17, 2023.

(51) Int. Cl. A61K 47/64; A61P 35/00

(52) U.S. Cl. CPC A61K 47/641; A61P 35/00

(57) ABSTRACT

This invention relates to multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the protein binding moiety is an inhibitor of apoptosis (IAP).

### CROSS-REFERENCE TO RELATED APPLICATIONS

This application claims the benefit of U.S. Provisional Applications No. 63/590,960, filed Oct. 17, 2023, which is hereby incorporated by reference.

### SEQUENCE LISTING STATEMENT

A Sequence Listing conforming to the rules of WIPO Standard ST.26 is hereby incorporated by reference. The Sequence Listing has been filed as an electronic document encoded as XML in UTF-8 text. The electronic document, created on May 11, 2024, is entitled “P-629783-USP1_ST26.xml” and is 20,805 bytes in size.

### FIELD OF THE INVENTION

The present invention relates to a multifunctional branched therapeutic agent comprising a target-homing moiety that is selective for cell entry, two or more target-binding moieties to engage in intracellular action, and a set of linker molecules that conjugate each of the moieties to a central core molecule. This novel therapeutic exploits the ability of a homing molecule to selectively bind to receptors of a specific cell type, promote cellular uptake, and then interact with an intracellular target through the chemical concept of avidity. In particular, the invention relates to multifunctional therapeutic peptide conjugates comprising a tumor-homing peptide, one or more target protein binding moieties, and linker molecules conjugating the tumor-homing peptide and the one or more target protein binding moieties to a branched reactive amino acid, wherein the protein binding moiety is an inhibitor of apoptosis (IAP). The invention also relates to methods for treating cancer comprising administering to a subject in need thereof a therapeutically effective amount of a multifunctional therapeutic peptide conjugate comprising a tumor-homing peptide, one or more target protein binding moieties, and linker molecules conjugating the tumor-homing peptide and the one or more target protein binding moieties to a branched reactive amino acid, wherein the protein binding moiety is an inhibitor of apoptosis (IAP).

### BACKGROUND OF THE INVENTION

Targeting intracellular proteins implicated in the pathogenesis of a variety diseases, including the leading causes of mortality and morbidity, such as cancers, cardiovascular diseases and even infectious disease, has been a long-standing challenge in the field of drug discovery and development. Many of these proteins have been termed “undruggable” due to their lack of traditional small-molecule binding sites, or because of issues related to drug delivery and specificity. This problem is further exacerbated when these target proteins are overexpressed or mutated in diseased cells, contributing to disease progression and therapeutic resistance.

Many of these undruggable targets interact with other biomolecules in situ suggesting that a peptidomimetic antagonist approach may be able to invoke a therapeutic effect. However, peptides and peptide-like molecules still suffer from cellular targeting, cellular uptake, and the possibility of weak binding to the target protein. A promising strategy to overcome these issues is covalently conjugate a number of active moieties and to then conjugate those active moieties to a homing peptide. While the homing peptide promotes cellular targeting and uptake, the additional number of active binding moieties increases the avidity of the binder for the target and in turn increases the therapeutic effect.

Accordingly, there remains a critical need for developing improved anticancer therapeutic compositions and enhanced methods for treating cancers.

### SUMMARY OF THE INVENTION

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the protein binding moiety is an inhibitor of apoptosis (IAP).

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a peptide sequence containing an RGD peptide motif consisting of the three amino acids Arg-Gly-Asp.

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1).

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2).

In still another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3).

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4).

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a peptide sequence containing a NGR motif consisting of the three amino acids Asn-Gly-Arg.

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a NGR motif consisting of the five amino acids Cys-Asn-Gly-Arg-Cys (SEQ ID NO: 5).

In still another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein tumor homing peptide comprises a cyclic peptide sequence consisting of the nine amino acid sequence Cys-Gly-Asn-Lys-Arg-Thr-Arg-Gly-Cys (SEQ ID NO: 6).

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7).

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the amino acids Cys-Arg-Glu-Lys-Ala (SEQ ID NO: 8).

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise one protein binding moiety consisting of a seven amino acid sequence AVPIAQK (SEQ ID NO: 10) from the N-terminus of the apoptosis mediator Smac/DIABLO protein.

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence [AIB]VPIAQK (SEQ ID NO: 11) from the N-terminus of the apoptosis mediator Smac/DIABLO protein, wherein [AIB] is 2-Aminoisobutyric acid.

In still another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence [AIB]VPI[AIB]QK (SEQ ID NO: 12) from the N-terminus of the apoptosis mediator Smac/DIABLO protein, wherein [AIB] is 2-Aminoisobutyric acid.

In still another aspect, provided herein are multifunctional therapeutic peptide conjugates represented by formula (T):

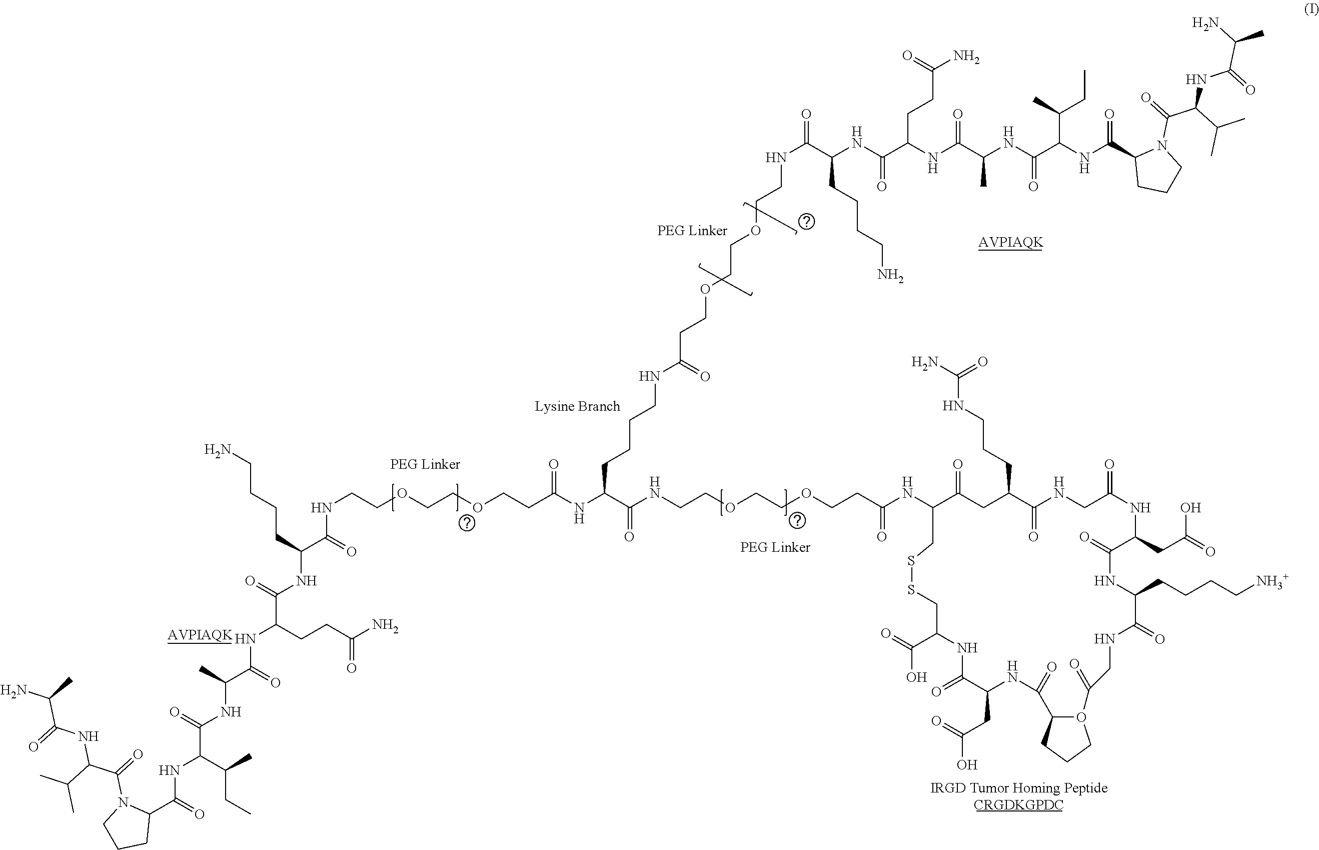

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence X₁X₂PIAQK (SEQ ID NO: 13) and X₁ is N-methyl alanine (MeA) and X₂ is tert-Leucine (Tle) (MeATlePIAQK (SEQ ID NO: 13)), from the N-terminus of the apoptosis mediator Smac/DIABLO protein.

In one aspect, provided herein are multifunctional therapeutic peptide conjugates represented by formula (II):

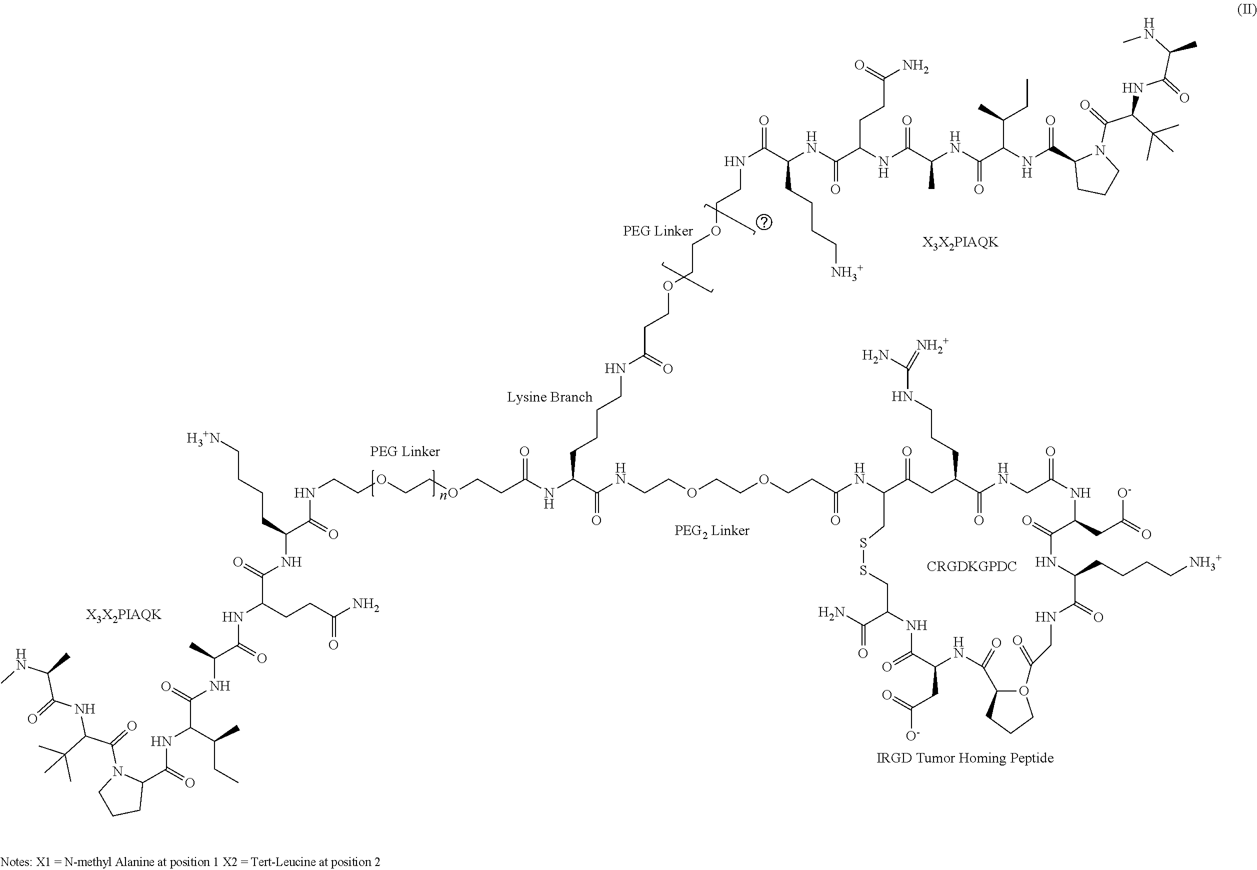

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein one of the two protein binding moiety consists of a seven amino acid sequence X₁X₂PFAQK (SEQ ID NO: 14), wherein X₁ is N-methyl alanine (MeA) and X₂ is tert-Leucine (Tle) (MeATlePFAQK (SEQ ID NO: 14)) and the second of the two protein binding moiety consists of a seven amino acid sequence X₁X₂PIAQK (SEQ ID NO: 13), wherein X₁ is N-methyl alanine (MeA) and X₂ is tert-Leucine (Tle) (MeATlePIAQK (SEQ ID NO: 13)), from the N-terminus of the apoptosis mediator Smac/DIABLO protein.

In one aspect, provided herein are multifunctional therapeutic peptide conjugates represented by formula (III):

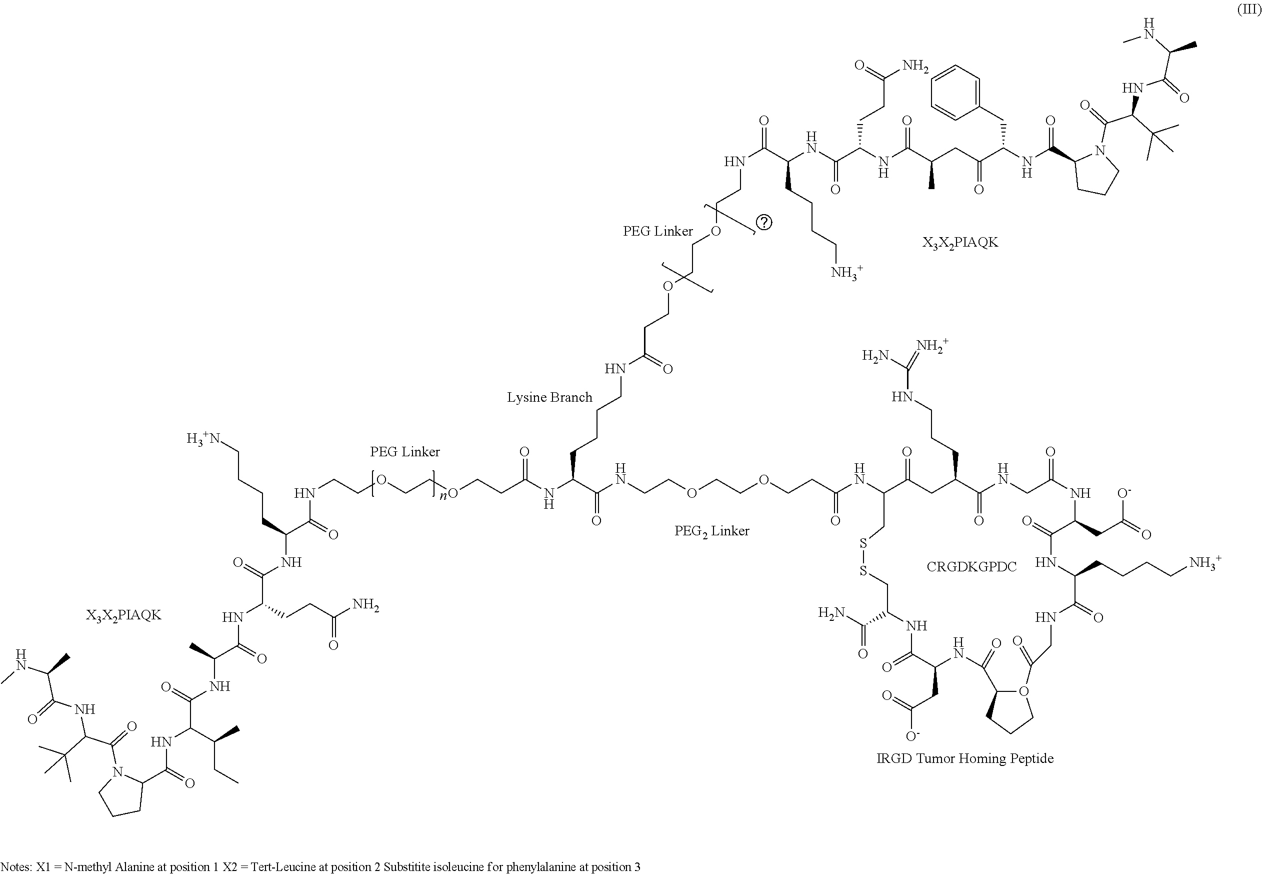

In another aspect, provided herein are methods for treating cancer comprising administering a to a subject in need thereof a therapeutically effective amount of a multifunctional therapeutic peptide conjugate compound to a subject in need thereof, the multifunctional therapeutic peptide conjugate compound comprising: a tumor homing peptide, a protein binding moiety and a branched reactive amino acid, wherein the tumor homing peptide is conjugated to a first linker, the protein binding moiety is conjugated to a second linker and the first and second linkers are conjugated to the branched reactive amino acid, wherein the protein binding moiety is an inhibitor of apoptosis (IAP).

In still another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a peptide sequence containing an RGD peptide motif consisting of the three amino acids Arg-Gly-Asp.

In one aspect, provided herein are provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1).

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2).

In still another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3).

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4).

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a peptide sequence containing a NGR motif consisting of the three amino acids Asn-Gly-Arg.

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a NGR motif consisting of the five amino acids Cys-Asn-Gly-Arg-Cys (SEQ ID NO: 5).

In still another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein tumor homing peptide comprises a cyclic peptide sequence consisting of the nine amino acid sequence Cys-Gly-Asn-Lys-Arg-Thr-Arg-Gly-Cys (SEQ ID NO: 6).

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7).

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the amino acids Cys-Arg-Glu-Lys-Ala (SEQ ID NO: 8).

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise one protein binding moiety consisting of a seven amino acid sequence AVPIAQK (SEQ ID NO: 10) from the N-terminus of the apoptosis mediator Smac/DIABLO protein.

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence [AIB]VPIAQK (SEQ ID NO: 11) from the N-terminus of the apoptosis mediator Smac/DIABLO protein, wherein [AIB] is 2-Aminoisobutyric acid.

In still another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence [AIB]VPI[AIB]QK (SEQ ID NO: 12) from the N-terminus of the apoptosis mediator Smac/DIABLO protein, wherein [AIB] is 2-Aminoisobutyric acid.

In another aspect, provided herein are methods for treating cancer comprising administering a to a subject in need thereof a therapeutically effective amount of a multifunctional therapeutic peptide conjugate represented by formula (I), as described herein.

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence X₁X₂PIAQK (SEQ ID NO: 13) and X₁ is N-methylated alanine (MeA) and X₂ is tert-Leucine (Tle) (i.e., MeATlePIAQK (SEQ ID NO: 13)), from the N-terminus of the apoptosis mediator Smac/DIABLO protein.

In another aspect, provided herein are methods for treating cancer comprising administering a to a subject in need thereof a therapeutically effective amount of a multifunctional therapeutic peptide conjugate represented by formula (II), as described herein.

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein one of the two protein binding moiety consists of a seven amino acid sequence X₁X₂PFAQK (SEQ ID NO: 14) (i.e., MeATlePFAQK (SEQ ID NO: 14)), wherein X₁ is N-methylated alanine (MeA) and X₂ is tert-Leucine (Tle) and the second of the two protein binding moiety consists of a seven amino acid sequence X₁X₂PIAQK (SEQ ID NO: 13) (i.e., MeATlePIAQK (SEQ ID NO: 13)), from the N-terminus of the apoptosis mediator Smac/DIABLO protein.

In another aspect, provided herein are methods for treating cancer comprising administering a to a subject in need thereof a therapeutically effective amount of a multifunctional therapeutic peptide conjugate represented by formula (III), as described herein.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The following drawing forms part of the present specification and are included to further demonstrate certain aspects of the present disclosure, the inventions of which can be better understood by reference to one or more of these drawings in combination with the detailed description of specific embodiments presented herein.

FIG. 1 depicts an embodiment of a multifunctional therapeutic peptide conjugate provided by the present disclosure.

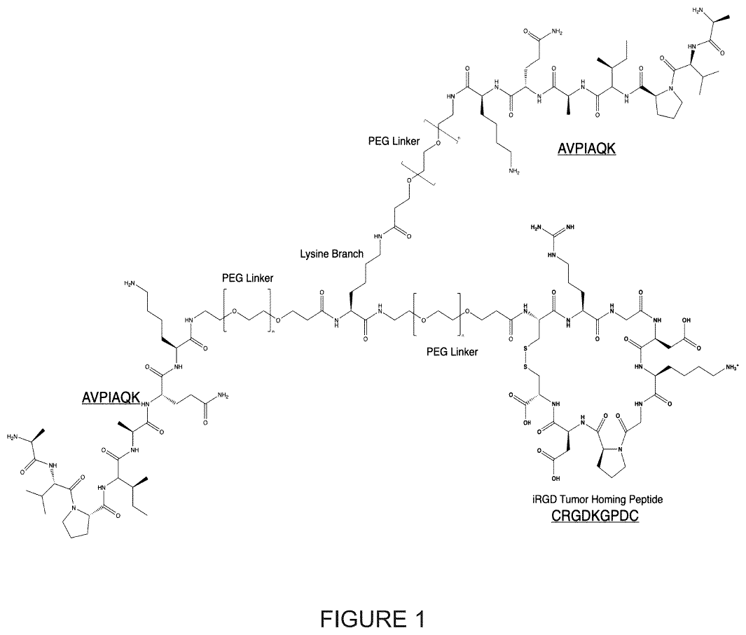

FIG. 2 depicts an embodiment of a multifunctional therapeutic peptide conjugate provided by the present disclosure in which each of the two XIAP binding domains, X₁X₂PIAQK (SEQ ID NO: 13), of the Smac/Diablo protein, is modified at amino acid sequence positions 1 and 2 to be N-methylated alanine and tert-leucine respectively, such that AVPIAQK (SEQ ID NO: 10) is modified to X₁X₂PIAQK (SEQ ID NO: 13), wherein X₁ is N-methylated alanine (MeA) at amino acid sequence position 1 and X₂ is tert-Leucine (Tle) at amino acid sequence position 2, i.e., to make MeATlePIAQK (SEQ ID NO: 13). These modifications improve proteolytic stability and increase binding affinity.

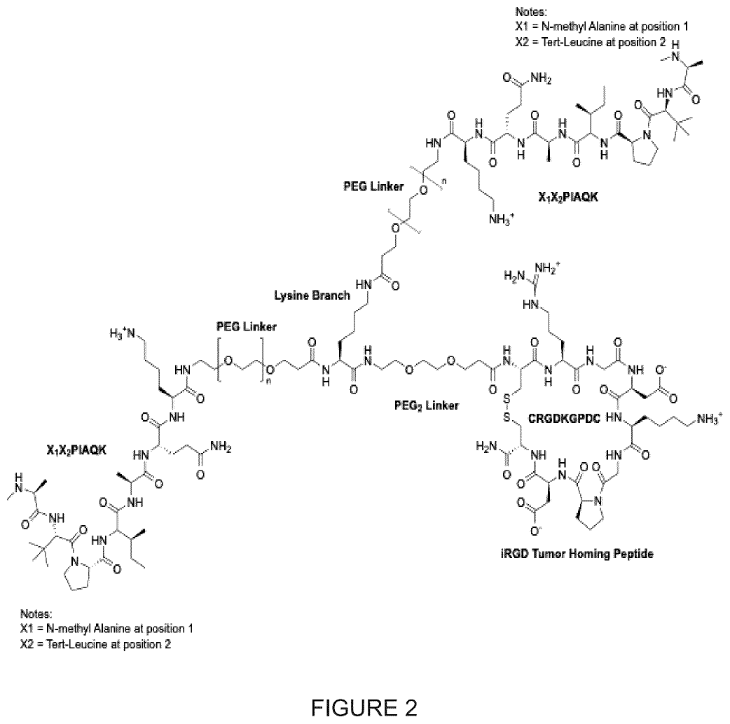

FIG. 3 depicts an embodiment of a multifunctional therapeutic peptide conjugate provided by the present disclosure in which one of the two fragment chains is modified at amino acid position 4 from isoleucine to phenylalanine (either one of the two chains may be modified): from X₁X₂PIAQK (SEQ ID NO: 13), wherein X₁ is N-methyl alanine (MeA) and X₂ is tert-Leucine (Tle), i.e., MeATlePIAQK (SEQ ID NO: 13) of the Smac/Diablo protein is modified at amino acid sequence position 4 from isoleucine (I) to phenylalanine (F), i.e., to X₁X₂PFAQK (SEQ ID NO: 14), wherein X₁ is and X₂ is N-methylated alanine (MeA) at amino acid sequence position 1 and X₂ is tert-Leucine (Tle) at amino acid sequence position 2, i.e., to MeATlePFAQK (SEQ ID NO: 14). This modification specifically biases one chain to bind the Bcl-2 domain of XIAP and the other domain to bind Bcl-3 domain of XIAP.

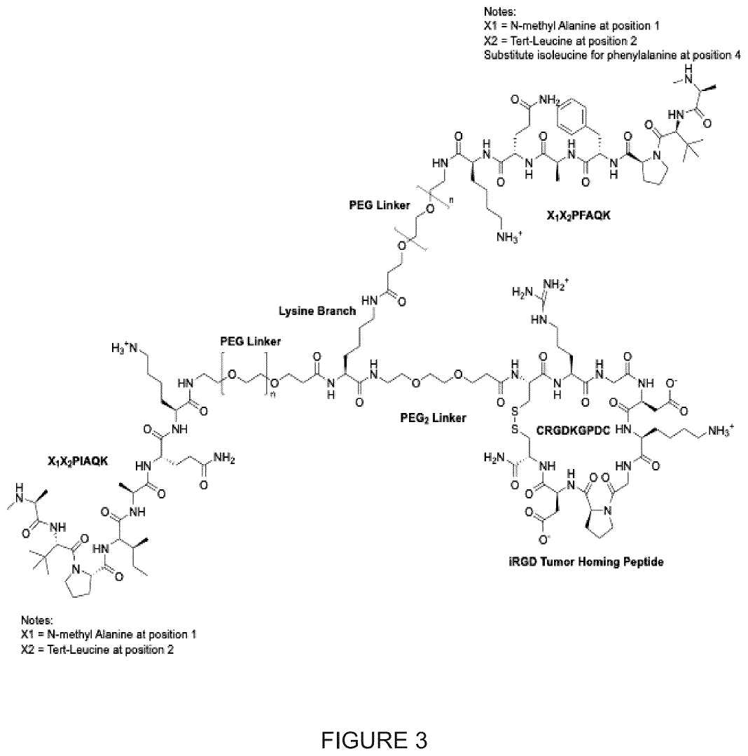

FIG. 4 depicts an embodiment of a a multifunctional therapeutic peptide conjugate provided by the present disclosure (BXL_2023.002) comprising a multivalent XIAP antagonist branched peptide conjugated to an iRGD tumor targeting peptide.

FIG. 5 is a plot of cell viability as a function of drug concentration for BXL_2023.002 (depicted in FIG. 4; where n=8) and showing specificity for pancreatic cancer cell line ASPC-1 (circles). Cell viability for the control HEK-293 cell line is also shown (squares).

It will be appreciated that for simplicity and clarity of illustration, elements shown in the figure have not necessarily been drawn to scale. For example, the dimensions of some of the elements may be exaggerated relative to other elements for clarity. Further, where considered appropriate, reference numerals may be repeated in the figure to indicate corresponding or analogous elements.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Numerous types of cancer occur in humans; however, each cancer cell has alterations that lead to the production of abnormal proteins responsible for uncontrolled cell growth and survival. Certain anti-cancer therapeutics bind to the such abnormal proteins, thereby impeding their activity. Nevertheless, some “undruggable” targets exist wherein the cancer-inducing proteins escape attempts to block their adverse effects.

Apoptosis is a form of programmed cell-death that occurs as a normal and controlled cellular process of an organism's growth or development to eliminate damaged or unwanted cells. Malfunctioning apoptosis is a characteristic of human cancers. Key regulators of cell death and survival are XIAP, cIAP1, and cIAP2, members of inhibitor of apoptosis (IAP) proteins.

Smac/DIABLO proteins are a family endogenous antagonists of inhibitors of apoptosis (IAP) proteins XIAP, cIAP1, and cIAP2.

The present invention provides a conjugate of a peptide moiety derived from the mature Smac/Diablo family of proteins and a tumor-homing peptide moiety known as iRGD, bridged by a reactive amino acid and polyethylene linkers, that offers the possibility to overcome the limitations of current peptide therapeutics, while delivering superior pharmacological benefits. The molecular linker may be PEG. The connection involves covalent bonding of the iRGD peptide to the α-amino group of the branched reactive amino acid via a molecular linker. In an embodiment the molecular linker is a PEG linker. The first seven amino acids from the N-terminus of the Smac/Diablo protein are then attached to both the α- and ε-amino groups of the lysine via a molecular linker creating a bifunctional therapeutic peptide conjugate. The peptide moieties are generally greater than 100 Da and less than 106.

The therapeutic conjugate described herein aim to resolve the issues of drug delivery and specificity of “undruggable” proteins.

In another aspect, the therapeutic peptide conjugate of this invention comprises an iRGD peptide, a branched reactive amino acid, and two fragments of the Smac/Diablo protein. Specifically, the conjugate consists of the iRGD peptide, a first linker, a branched reactive amino acid, a second and third linker, two fragments of the Smac/Diablo protein each consisting of the seven amino acid sequence AVPIAQK (SEQ ID NO: 10) from its N-terminus, wherein the first, second, and third linkers are each conjugated to the branched reactive amino acid by a molecular linker. A branched reactive amino acid is defined herein as an amino acid residue having two active branching sites. Each active branching site is an amine group, an azido group, a thiol group or a diamine group having two amines. In a particular embodiment, the branched reactive amino acid is a lysine containing two primary amine groups. In an embodiment, the lysine is L-lysine or D-lysine or an azido-lysine, i.e., 6-azido-L-lysine or 6-azido-D-lysine. In another embodiment, the reactive amino acid is a cysteine, which contains a reactive thiol (or “sulfhydryl”) group. In an embodiment, the reactive amino acid is a 5-azido-L-ornithine or 5-azido-d-ornithine. The each molecular linker may be polyethylene glycol (PEG). The PEG linkers may be the same PEG or different a different PEG. In an embodiment, the first linker and the second linker is an identical or different polyethylene glycol (PEG) selected from the group consisting of amino-PEG2, amino-PEG3, amino-PEG4, amino-PEG6, amino-PEG8, amino-PEG10, amino-PEG12 and amino-PEG20.

In an embodiment of the herein described multifunctional therapeutic peptide conjugates, the multifunctional therapeutic peptide conjugate comprises a tumor homing peptide, at least one protein binding moiety and a branched reactive amino acid, wherein the tumor homing peptide is conjugated to a first linker, the at least one protein binding moiety is conjugated to a second linker and the first and second linkers are conjugated to the branched reactive amino acid, wherein the at least one protein binding moiety is an inhibitor of apoptosis (IAP). In certain embodiments, the at least one protein binding moiety consists of a seven amino acid sequence AVPIAQK (SEQ ID NO: 10) from the N-terminus of the apoptosis mediator Smac/DIABLO protein. In some embodiments, the at least one protein binding moiety consists of a seven amino acid sequence 2-Aminoisobutyric acid [AIB]VPIAQK (SEQ ID NO: 11) from the N-terminus of the apoptosis mediator Smac/DIABLO protein. In an embodiment, the at least one protein binding moiety consists of a seven amino acid sequence [AIB]VPI[AIB]QK (SEQ ID NO: 12) from the N-terminus of the apoptosis mediator Smac/DIABLO protein. In a particular embodiment, the multifunctional therapeutic peptide conjugate comprises two protein binding moieties, wherein each protein binding moiety consists of a seven amino acid sequence AVPIAQK (SEQ ID NO: 10) from the N-terminus of the apoptosis mediator Smac/DIABLO protein. In various embodiments, the multifunctional therapeutic peptide conjugate comprises two protein binding moieties, wherein each protein binding moiety consists of a seven amino acid sequence 2-Aminoisobutyric acid [AIB]VPIAQK (SEQ ID NO: 11) from the N-terminus of the apoptosis mediator Smac/DIABLO protein. In an embodiment, the multifunctional therapeutic peptide conjugate comprises two protein binding moieties, wherein each protein binding moiety consists of a seven amino acid sequence [AIB]VPI[AIB]QK (SEQ ID NO: 12) from the N-terminus of the apoptosis mediator Smac/DIABLO protein. In another embodiment, the multifunctional therapeutic peptide conjugate comprises three protein binding moieties, wherein each protein binding moiety consists of a seven amino acid sequence AVPIAQK (SEQ ID NO: 10) from the N-terminus of the apoptosis mediator Smac/DIABLO protein. In some embodiments, the multifunctional therapeutic peptide conjugate comprises three protein binding moieties, wherein each protein binding moiety consists of a seven amino acid sequence 2-Aminoisobutyric acid [AIB]VPIAQK (SEQ ID NO: 11) from the N-terminus of the apoptosis mediator Smac/DIABLO protein. In certain embodiments, the multifunctional therapeutic peptide conjugate comprises three protein binding moieties, wherein each protein binding moiety consists of a seven amino acid sequence [AIB]VPI[AIB]QK (SEQ ID NO: 12) from the N-terminus of the apoptosis mediator Smac/DIABLO protein.

The invention relates to multifunctional therapeutic peptide conjugates comprising a tumor-homing peptide, one or more target protein binding moieties, and linker molecules conjugating the tumor-homing peptide and the one or more target protein binding moieties to a branched reactive amino acid, wherein the protein binding moiety is an inhibitor of apoptosis (IAP). The invention also relates to methods for treating cancer comprising administering to a subject in need thereof a therapeutically effective amount of a multifunctional therapeutic peptide conjugate comprising a tumor-homing peptide, one or more target protein binding moieties, and linker molecules conjugating the tumor-homing peptide and the one or more target protein binding moieties to a branched reactive amino acid, wherein the protein binding moiety is an inhibitor of apoptosis (IAP). In particular embodiments, the invention relates to a therapeutic multivalent SMAC/Diablo protein peptidomimetic covalently linked to a tumor-homing peptide, wherein the tumor-homing peptide and the N-terminal seven amino acid sequence AVPIAQK (SEQ ID NO: 10) of the SMAC/Diablo protein are each conjugated to a linker molecule and each linker molecule is connected to a branched reactive amino acid.

In one aspect, the present invention provides an improved bifunctional therapeutic peptide conjugate provided herein comprises an N-terminal seven amino acid sequence AVPIAQK (SEQ ID NO: 10) of the SMAC/Diablo protein and the iRGD peptide, connected via a central branched reactive amino acid, wherein the iRGD peptide and the N-terminal seven amino acid sequence AVPIAQK (SEQ ID NO: 10) of the SMAC/Diablo protein are each conjugated to a linker molecule and each linker molecule is connected to the central branched reactive amino acid. In an embodiment, the central branched reactive amino acid is lysine. In an embodiment, the molecular linker is polyethylene glycol (PEG).

In another aspect, the present invention provides a multifunctional therapeutic peptide conjugate comprising: a tumor homing peptide, a protein binding moiety and a branched reactive amino acid, wherein the tumor homing peptide is conjugated to a first linker, the protein binding moiety is conjugated to a second linker and the first and second linkers are conjugated to the branched reactive amino acid, wherein the protein binding moiety is an inhibitor of apoptosis (IAP). In an embodiment of the multifunctional therapeutic peptide conjugate, the tumor homing peptide is the cyclic peptide iRGD having amino acid sequence CRGD[K/R]GP[D/E]C, i.e., Cys-Arg-Gly-Asp-Lys-Gly-Pro-Asp-Cys (SEQ ID NO: 1) and Cys-Arg-Gly-Asp-Arg-Gly-Pro-Glu-Cys (SEQ ID NO: 4). In an embodiment, the tumor homing peptide is the cyclic peptide iRGD having amino acid sequence Cys-Asn-Gly-Arg-Cys (SEQ ID NO: 5). In another embodiment, the tumor homing peptide is the cyclic peptide iRGD having amino acid sequence Cys-Gly-Asn-Lys-Arg-Thr-Arg-Gly-Cys (SEQ ID NO: 6). In some embodiments, the tumor homing peptide is the cyclic peptide iRGD having amino acid sequence Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In an embodiment, the tumor homing peptide is the cyclic peptide iRGD having amino acid sequence Cys-Arg-Glu-Lys-Ala (SEQ ID NO: 8). In another embodiment, the tumor homing peptide is the cyclic peptide iRGD having amino acid sequence Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9). In an embodiment, the branched reactive amino acid is a lysine, a cysteine, an azido-lysine or an azido-ornithine. In a particular embodiment, the branched reactive amino acid is lysine. In another particular embodiment, the branched reactive amino acid is cysteine. In an embodiment, the lysine is L-lysine or D-lysine or an azido-lysine, i.e., 6-azido-L-lysine or 6-azido-D-lysine. In an embodiment, the branched reactive amino acid is azido-Lysine. In an embodiment, the reactive amino acid is a 5-azido-L-ornithine or 5-azido-d-ornithine. In some embodiments, the multifunctional therapeutic peptide conjugate comprises one protein binding moiety consisting of a seven amino acid sequence AVPIAQK (SEQ ID NO: 10) from the N-terminus of the apoptosis mediator Smac/DIABLO protein. In an embodiment, the multifunctional therapeutic peptide conjugate comprises one protein binding moiety consisting of a seven amino acid sequence [AIB]VPIAQK (SEQ ID NO: 11), wherein [AIB] is 2-Aminoisobutyric acid. In another embodiment, the multifunctional therapeutic peptide conjugate comprises one protein binding moiety consisting of a seven amino acid sequence [AIB]VPI[AIB]QK (SEQ ID NO: 12), wherein [AIB] is 2-Aminoisobutyric acid. In some embodiments, the multifunctional therapeutic peptide conjugate comprises two protein binding moieties, wherein each protein binding moiety consists of a seven amino acid sequence AVPIAQK (SEQ ID NO: 10) from the N-terminus of the apoptosis mediator Smac/DIABLO protein. In some embodiments, the first and second linker are the same. In certain embodiments, the first and second linker are different linker molecules. In an embodiment of the multifunctional therapeutic peptide conjugate, the molecular linker is polyethylene glycol (PEG). In a particular embodiment, the first and second polyethylene glycol (PEG) molecular linker, includes but is not limited to amino-PEG2, amino-PEG3, amino-PEG4, amino-PEG6, amino-PEG8, amino-PEG10, amino-PEG12, and/or amino-PEG20. In some embodiments, the first and second linker are poly(glycerols) (PGs), poly(oxazolines) (POX), poly(hydroxypropyl methacrylate) (PHPMA), poly(2-hydroxyethyl methacrylate) (PHEMA), poly(N-(2-hydroxypropyl) methacrylamide) (HPMA), poly(vinylpyrrolidone) (PVP), poly(N,N-dimethyl acrylamide) (PDMA), and/or poly(N-acryloylmorpholine) (PAcM). In an embodiment of the multifunctional therapeutic peptide conjugate, the linker enhances the stability and bioavailability of the conjugate. In certain embodiments, the multifunctional therapeutic peptide conjugate is for use in the treatment of cancer.

In another aspect, the present the invention provides an improved bifunctional therapeutic peptide conjugate comprising the N-terminus of the Smac/Diablo protein and the iRGD peptide, wherein the N-terminus of the Smac/Diablo protein and the iRGD peptide are each connected to lysine by polyethylene glycol (PEG).

In one aspect, the present invention provides an improved multifunctional therapeutic peptide conjugate comprising two N-terminal seven amino acid sequences AVPIAQK (SEQ ID NO: 10) of the SMAC/Diablo protein and the iRGD peptide, wherein each N-terminal seven amino acid sequence AVPIAQK (SEQ ID NO: 10) of the Smac/Diablo protein and the iRGD peptide are connected to a central branched reactive amino acid by a linker molecule and each linker molecule is connected to the central branched reactive amino acid. In an embodiment, the central branched reactive amino acid is lysine. In an embodiment, the molecular linker is polyethylene glycol (PEG).

In another aspect, the present invention provides an improved multifunctional therapeutic peptide conjugate comprising two N-terminal seven amino acid sequences AVPIAQK (SEQ ID NO: 10) of the SMAC/Diablo protein and the iRGD peptide, wherein each N-terminal seven amino acid sequence AVPIAQK (SEQ ID NO: 10) of the Smac/Diablo protein and the iRGD peptide are connected to a lysine by polyethylene glycol (PEG).

In a further aspect, the present the invention provides a method for treating cancer comprising administering a therapeutically effective amount of a multifunctional therapeutic peptide conjugate to a subject in need thereof, the multifunctional therapeutic peptide conjugate comprising: a tumor homing peptide, a protein binding moiety and a branched reactive amino acid, wherein the tumor homing peptide is conjugated to a first linker, the protein binding moiety is conjugated to a second linker and the first and second linkers are conjugated to the branched reactive amino acid, wherein the protein binding moiety is an inhibitor of apoptosis (IAP). In an embodiment of said method, the tumor homing peptide is the cyclic peptide iRGD having amino acid sequence CRGD[K/R]GP[D/E]C (SEQ ID NO: 1) and (SEQ ID NO: 4), i.e., Cys-Arg-Gly-Asp-Lys-Gly-Pro-Asp-Cys (SEQ ID NO: 1) and Cys-Arg-Gly-Asp-Arg-Gly-Pro-Glu-Cys (SEQ ID NO: 4).

In an embodiment, the branched reactive amino acid is lysine. In some embodiments of the herein provided method, the multifunctional therapeutic peptide conjugate comprises one protein binding moiety consisting of a seven amino acid sequence AVPIAQK (SEQ ID NO: 10) from the N-terminus of the apoptosis mediator Smac/DIABLO protein. In certain embodiments of the herein provided method, the multifunctional therapeutic peptide conjugate comprises two protein binding moieties, wherein each protein binding moiety consists of a seven amino acid sequence AVPIAQK (SEQ ID NO: 10) from the N-terminus of the apoptosis mediator Smac/DIABLO protein. In some embodiments of said methods, the molecular linker is polyethylene glycol (PEG).

In one aspect, the present the invention provides a method for treating cancer comprising administering a to a subject in need thereof a therapeutically effective amount of an improved bifunctional therapeutic peptide conjugate comprising the N-terminus of the Smac/Diablo protein consisting of seven amino acid sequence AVPIAQK (SEQ ID NO: 10) and the iRGD peptide, wherein the N-terminus of the Smac/Diablo protein and the iRGD peptide are connected to a branched reactive amino acid by a molecular linker. In an embodiment of the provided methods, the branched reactive amino acid is lysine. In an embodiment, the molecular linker is polyethylene glycol (PEG).

In another aspect, the present the invention provides a method for treating cancer comprising administering to a subject in need thereof a therapeutically effective amount of an improved multifunctional therapeutic peptide conjugate comprising two N-termini of the Smac/Diablo protein, each N-terminal sequence consisting of the seven amino acid sequence AVPIAQK (SEQ ID NO: 10) of the Smac/Diablo protein and the iRGD peptide, wherein the two N-termini and the iRGD peptide are each connected to a branched reactive amino acid by a molecular linker. In an embodiment of the provided methods, the branched reactive amino acid is lysine. In an embodiment, the molecular linker is polyethylene glycol (PEG).

In an embodiment of the herein described methods for treating cancer, the multifunctional therapeutic peptide conjugate comprises a tumor homing peptide, at least one protein binding moiety and a branched reactive amino acid, wherein the tumor homing peptide is conjugated to a first linker, the at least one protein binding moiety is conjugated to a second linker and the first and second linkers are conjugated to the branched reactive amino acid, wherein the at least one protein binding moiety is an inhibitor of apoptosis (IAP). In certain embodiments, the at least one protein binding moiety consists of a seven amino acid sequence AVPIAQK (SEQ ID NO: 10) from the N-terminus of the apoptosis mediator Smac/DIABLO protein. In some embodiments, the at least one protein binding moiety consists of a seven amino acid sequence 2-Aminoisobutyric acid [AIB]VPIAQK (SEQ ID NO: 11) from the N-terminus of the apoptosis mediator Smac/DIABLO protein. In an embodiment, the at least one protein binding moiety consists of a seven amino acid sequence [AIB]VPI[AIB]QK (SEQ ID NO: 12) from the N-terminus of the apoptosis mediator Smac/DIABLO protein. In a particular embodiment, the multifunctional therapeutic peptide conjugate comprises two protein binding moieties, wherein each protein binding moiety consists of a seven amino acid sequence AVPIAQK (SEQ ID NO: 10) from the N-terminus of the apoptosis mediator Smac/DIABLO protein. In various embodiments, the multifunctional therapeutic peptide conjugate comprises two protein binding moieties, wherein each protein binding moiety consists of a seven amino acid sequence 2-Aminoisobutyric acid [AIB]VPIAQK (SEQ ID NO: 11) from the N-terminus of the apoptosis mediator Smac/DIABLO protein. In an embodiment, the multifunctional therapeutic peptide conjugate comprises two protein binding moieties, wherein each protein binding moiety consists of a seven amino acid sequence [AIB]VPI[AIB]QK (SEQ ID NO: 12) from the N-terminus of the apoptosis mediator Smac/DIABLO protein. In another embodiment, the multifunctional therapeutic peptide conjugate comprises three protein binding moieties, wherein each protein binding moiety consists of a seven amino acid sequence AVPIAQK (SEQ ID NO: 10) from the N-terminus of the apoptosis mediator Smac/DIABLO protein. In some embodiments, the multifunctional therapeutic peptide conjugate comprises three protein binding moieties, wherein each protein binding moiety consists of a seven amino acid sequence 2-Aminoisobutyric acid [AIB]VPIAQK (SEQ ID NO: 11) from the N-terminus of the apoptosis mediator Smac/DIABLO protein. In certain embodiments, the multifunctional therapeutic peptide conjugate comprises three protein binding moieties, wherein each protein binding moiety consists of a seven amino acid sequence [AIB]VPI[AIB]QK (SEQ ID NO: 12) from the N-terminus of the apoptosis mediator Smac/DIABLO protein.

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the protein binding moiety is an inhibitor of apoptosis (IAP).

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a peptide sequence containing an RGD peptide motif consisting of the three amino acids Arg-Gly-Asp.

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1).

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2).

In still another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3).

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4).

In embodiments of the above-described multifunctional therapeutic peptide conjugate compounds comprising an active cellular targeting moiety (A) having a cyclic peptide sequence, the cyclic peptide sequence containing the cyclic iRGD peptide motif is cyclized via a disulfide bond between cysteine at residue position 1 and cysteine at residue position 9.

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a peptide sequence containing a NGR motif consisting of the three amino acids Asn-Gly-Arg.

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a NGR motif consisting of the five amino acids Cys-Asn-Gly-Arg-Cys (SEQ ID NO: 5).

In certain embodiments of the above-described multifunctional therapeutic peptide conjugate compounds comprising an active cellular targeting moiety (A) having a cyclic peptide sequence containing the NGR motif, the cyclic peptide sequence is cyclized with a disulfide bond between cysteine at residue position 1 and cysteine at residue position 5. In an embodiment of the above-described multifunctional therapeutic peptide conjugate compounds comprising an active cellular targeting moiety (A) having a cyclic peptide sequence containing the NGR motif, the active cellular targeting moiety targets the extracellular enzyme Aminopeptidase N (CD13).

In still another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein tumor homing peptide comprises a cyclic peptide sequence consisting of the nine amino acid sequence Cys-Gly-Asn-Lys-Arg-Thr-Arg-Gly-Cys (SEQ ID NO: 6).

In an embodiment of the above-described multifunctional therapeutic peptide conjugate compounds comprising the cyclic peptide sequence consisting of the nine amino acid sequence Cys-Gly-Asn-Lys-Arg-Thr-Arg-Gly-Cys, the cyclic peptide is cyclized with a disulfide bond between cysteine at residue position 1 and cysteine at residue position 9. In some embodiments of said multifunctional therapeutic peptide conjugate compounds, the active cellular targeting moiety targets the cellular receptor p32/gC1qR.

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In an embodiment of said multifunctional therapeutic peptide conjugate compounds, the active cellular targeting moiety targets the Nucleolin cellular receptor.

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the amino acids Cys-Arg-Glu-Lys-Ala (SEQ ID NO: 8). In an embodiment of said multifunctional therapeutic peptide conjugate compounds, the active cellular targeting moiety targets Fibrin-fibronectin complexes.

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9). In an embodiment of said multifunctional therapeutic peptide conjugate compounds, the active cellular targeting moiety targets the cellular receptor GRP78.

In an embodiment of the above-described multifunctional therapeutic peptide conjugate compounds, the branched reactive amino acid is a lysine, a cysteine, an azido-lysine or an azido-ornithine. In a particular embodiment, the branched reactive amino acid is lysine. In another embodiment, the branched reactive amino acid is cysteine. In some embodiments, the branched reactive amino acid is azido-lysine. In an embodiment, the lysine is L-lysine or D-lysine or an azido-lysine, i.e., 6-azido-L-lysine or 6-azido-D-lysine. In an embodiment, the reactive amino acid is a 5-azido-L-ornithine or 5-azido-d-ornithine.

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise one protein binding moiety consisting of a seven amino acid sequence AVPIAQK (SEQ ID NO: 10) from the N-terminus of the apoptosis mediator Smac/DIABLO protein. In an embodiment, the branched reactive amino acid is a lysine, a cysteine, an azido-lysine or an azido-ornithine. In a particular embodiment, the branched reactive amino acid is lysine. In another embodiment, the branched reactive amino acid is cysteine. In some embodiments, the branched reactive amino acid is azido-lysine. In an embodiment, the lysine is L-lysine or D-lysine or an azido-lysine, i.e., 6-azido-L-lysine or 6-azido-D-lysine. In an embodiment, the reactive amino acid is a 5-azido-L-ornithine or 5-azido-d-ornithine.

In an embodiment, the tumor homing peptide comprises an active cellular targeting moiety (A) having a peptide sequence containing an RGD peptide motif consisting of the three amino acids Arg-Gly-Asp. In another embodiment, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2). In some embodiments, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4). In another embodiment, the cyclic peptide sequence containing the cyclic iRGD peptide motif is cyclized via a disulfide bond between cysteine at residue position 1 and cysteine at residue position 9. In some embodiments, the tumor homing peptide comprises an active cellular targeting moiety (A) having a peptide sequence containing a NGR motif consisting of the three amino acids Asn-Gly-Arg. In an embodiment, the tumor homing peptide comprises an active cellular targeting moiety (A) having a cyclic peptide sequence containing a NGR motif consisting of the five amino acids Cys-Asn-Gly-Arg-Cys (SEQ ID NO: 5). In an embodiment, the cyclic peptide sequence containing the NGR motif is cyclized with a disulfide bond between cysteine at residue position 1 and cysteine at residue position 5. In another embodiment, the tumor homing peptide comprises a cyclic peptide sequence consisting of the nine amino acid sequence Cys-Gly-Asn-Lys-Arg-Thr-Arg-Gly-Cys (SEQ ID NO: 6). In an embodiment, the cyclic peptide is cyclized with a disulfide bond between cysteine at residue position 1 and cysteine at residue position 9. In another embodiment, the tumor homing peptide comprises a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In some embodiments, the tumor homing peptide has a peptide sequence consisting of the amino acids Cys-Arg-Glu-Lys-Ala (SEQ ID NO: 8). In another embodiment, the tumor homing peptide comprises a peptide sequence consisting of the amino acids Cys-Arg-Glu-Lys-Ala (SEQ ID NO: 8). In an embodiment, the tumor homing peptide comprises a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise one protein binding moiety consisting of a seven amino acid sequence consists of a seven amino acid sequence [AIB]VPIAQK (SEQ ID NO: 11) from the N-terminus of the apoptosis mediator Smac/DIABLO protein, wherein [AIB] is 2-Aminoisobutyric acid. In an embodiment, the branched reactive amino acid is a lysine, a cysteine, an azido-lysine or an azido-ornithine. In a particular embodiment, the branched reactive amino acid is lysine. In another embodiment, the branched reactive amino acid is cysteine. In some embodiments, the branched reactive amino acid is azido-lysine. In an embodiment, the lysine is L-lysine or D-lysine or an azido-lysine, i.e., 6-azido-L-lysine or 6-azido-D-lysine. In an embodiment, the reactive amino acid is a 5-azido-L-ornithine or 5-azido-d-ornithine.

In an embodiment, the tumor homing peptide comprises an active cellular targeting moiety (A) having a peptide sequence containing an RGD peptide motif consisting of the three amino acids Arg-Gly-Asp. In another embodiment, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2). In some embodiments, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4). In another embodiment, the cyclic peptide sequence containing the cyclic iRGD peptide motif is cyclized via a disulfide bond between cysteine at residue position 1 and cysteine at residue position 9. In some embodiments, the tumor homing peptide comprises an active cellular targeting moiety (A) having a peptide sequence containing a NGR motif consisting of the three amino acids Asn-Gly-Arg. In an embodiment, the tumor homing peptide comprises an active cellular targeting moiety (A) having a cyclic peptide sequence containing a NGR motif consisting of the five amino acids Cys-Asn-Gly-Arg-Cys (SEQ ID NO: 5). In an embodiment, the cyclic peptide sequence containing the NGR motif is cyclized with a disulfide bond between cysteine at residue position 1 and cysteine at residue position 5. In another embodiment, the tumor homing peptide comprises a cyclic peptide sequence consisting of the nine amino acid sequence Cys-Gly-Asn-Lys-Arg-Thr-Arg-Gly-Cys (SEQ ID NO: 6). In an embodiment, the cyclic peptide is cyclized with a disulfide bond between cysteine at residue position 1 and cysteine at residue position 9. In another embodiment, the tumor homing peptide comprises a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In some embodiments, the tumor homing peptide has a peptide sequence consisting of the amino acids Cys-Arg-Glu-Lys-Ala (SEQ ID NO: 5). In another embodiment, the tumor homing peptide comprises a peptide sequence consisting of the amino acids Cys-Arg-Glu-Lys-Ala (SEQ ID NO: 5). In an embodiment, the tumor homing peptide comprises a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9). In an embodiment, the tumor homing peptide comprises an active cellular targeting moiety (A) having a peptide sequence containing an RGD peptide motif consisting of the three amino acids Arg-Gly-Asp. In another embodiment, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2). In some embodiments, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4). In another embodiment, the cyclic peptide sequence containing the cyclic iRGD peptide motif is cyclized via a disulfide bond between cysteine at residue position 1 and cysteine at residue position 9. In some embodiments, the tumor homing peptide comprises an active cellular targeting moiety (A) having a peptide sequence containing a NGR motif consisting of the three amino acids Asn-Gly-Arg. In an embodiment, the tumor homing peptide comprises an active cellular targeting moiety (A) having a cyclic peptide sequence containing a NGR motif consisting of the five amino acids Cys-Asn-Gly-Arg-Cys (SEQ ID NO: 5). In an embodiment, the cyclic peptide sequence containing the NGR motif is cyclized with a disulfide bond between cysteine at residue position 1 and cysteine at residue position 5. In another embodiment, the tumor homing peptide comprises a cyclic peptide sequence consisting of the nine amino acid sequence Cys-Gly-Asn-Lys-Arg-Thr-Arg-Gly-Cys (SEQ ID NO: 6). In an embodiment, the cyclic peptide is cyclized with a disulfide bond between cysteine at residue position 1 and cysteine at residue position 9. In another embodiment, the tumor homing peptide comprises a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In some embodiments, the tumor homing peptide has a peptide sequence consisting of the amino acids Cys-Arg-Glu-Lys-Ala (SEQ ID NO: 8). In another embodiment, the tumor homing peptide comprises a peptide sequence consisting of the amino acids Cys-Arg-Glu-Lys-Ala. In an embodiment, the tumor homing peptide comprises a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise one protein binding moiety consisting of a seven amino acid sequence [AIB]VPI[AIB]QK (SEQ ID NO: 12) from the N-terminus of the apoptosis mediator Smac/DIABLO protein, wherein [AIB] is 2-Aminoisobutyric acid. In an embodiment, the branched reactive amino acid is a lysine, a cysteine, an azido-lysine or an azido-ornithine. In a particular embodiment, the branched reactive amino acid is lysine. In another embodiment, the branched reactive amino acid is cysteine. In some embodiments, the branched reactive amino acid is azido-lysine. In an embodiment, the lysine is L-lysine or D-lysine or an azido-lysine, i.e., 6-azido-L-lysine or 6-azido-D-lysine. In an embodiment, the reactive amino acid is a 5-azido-L-ornithine or 5-azido-d-ornithine. In an embodiment, the tumor homing peptide comprises an active cellular targeting moiety (A) having a peptide sequence containing an RGD peptide motif consisting of the three amino acids Arg-Gly-Asp. In another embodiment, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2). In some embodiments, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4). In another embodiment, the cyclic peptide sequence containing the cyclic iRGD peptide motif is cyclized via a disulfide bond between cysteine at residue position 1 and cysteine at residue position 9. In some embodiments, the tumor homing peptide comprises an active cellular targeting moiety (A) having a peptide sequence containing a NGR motif consisting of the three amino acids Asn-Gly-Arg. In an embodiment, the tumor homing peptide comprises an active cellular targeting moiety (A) having a cyclic peptide sequence containing a NGR motif consisting of the five amino acids Cys-Asn-Gly-Arg-Cys (SEQ ID NO: 5). In an embodiment, the cyclic peptide sequence containing the NGR motif is cyclized with a disulfide bond between cysteine at residue position 1 and cysteine at residue position 5. In another embodiment, the tumor homing peptide comprises a cyclic peptide sequence consisting of the nine amino acid sequence Cys-Gly-Asn-Lys-Arg-Thr-Arg-Gly-Cys (SEQ ID NO: 6). In an embodiment, the cyclic peptide is cyclized with a disulfide bond between cysteine at residue position 1 and cysteine at residue position 9. In another embodiment, the tumor homing peptide comprises a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In some embodiments, the tumor homing peptide has a peptide sequence consisting of the amino acids Cys-Arg-Glu-Lys-Ala (SEQ ID NO: 8). In another embodiment, the tumor homing peptide comprises a peptide sequence consisting of the amino acids Cys-Arg-Glu-Lys-Ala. In an embodiment, the tumor homing peptide comprises a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence [AIB]VPIAQK (SEQ ID NO: 11) from the N-terminus of the apoptosis mediator Smac/DIABLO protein, wherein [AIB] is 2-Aminoisobutyric acid. In an embodiment, the branched reactive amino acid is a lysine, a cysteine, an azido-lysine or an azido-ornithine. In a particular embodiment, the branched reactive amino acid is lysine. In another embodiment, the branched reactive amino acid is cysteine. In some embodiments, the branched reactive amino acid is azido-lysine. In an embodiment, the lysine is L-lysine or D-lysine or an azido-lysine, i.e., 6-azido-L-lysine or 6-azido-D-lysine. In an embodiment, the reactive amino acid is a 5-azido-L-ornithine or 5-azido-d-ornithine.

In an embodiment, the branched reactive amino acid is lysine, cysteine or azido-lysine. In a particular embodiment, the branched reactive amino acid is lysine. In another embodiment, the branched reactive amino acid is cysteine. In some embodiments, the branched reactive amino acid is azido-lysine. In an embodiment, the lysine is L-lysine or D-lysine or an azido-lysine, i.e., 6-azido-L-lysine or 6-azido-D-lysine. In an embodiment, the reactive amino acid is a 5-azido-L-ornithine or 5-azido-d-ornithine. In an embodiment, the tumor homing peptide comprises an active cellular targeting moiety (A) having a peptide sequence containing an RGD peptide motif consisting of the three amino acids Arg-Gly-Asp. In another embodiment, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2). In some embodiments, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4). In another embodiment, the cyclic peptide sequence containing the cyclic iRGD peptide motif is cyclized via a disulfide bond between cysteine at residue position 1 and cysteine at residue position 9. In some embodiments, the tumor homing peptide comprises an active cellular targeting moiety (A) having a peptide sequence containing a NGR motif consisting of the three amino acids Asn-Gly-Arg. In an embodiment, the tumor homing peptide comprises an active cellular targeting moiety (A) having a cyclic peptide sequence containing a NGR motif consisting of the five amino acids Cys-Asn-Gly-Arg-Cys (SEQ ID NO: 5). In an embodiment, the cyclic peptide sequence containing the NGR motif is cyclized with a disulfide bond between cysteine at residue position 1 and cysteine at residue position 5. In another embodiment, the tumor homing peptide comprises a cyclic peptide sequence consisting of the nine amino acid sequence Cys-Gly-Asn-Lys-Arg-Thr-Arg-Gly-Cys (SEQ ID NO: 6). In an embodiment, the cyclic peptide is cyclized with a disulfide bond between cysteine at residue position 1 and cysteine at residue position 9. In another embodiment, the tumor homing peptide comprises a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In some embodiments, the tumor homing peptide has a peptide sequence consisting of the amino acids Cys-Arg-Glu-Lys-Ala. In another embodiment, the tumor homing peptide comprises a peptide sequence consisting of the amino acids Cys-Arg-Glu-Lys-Ala (SEQ ID NO: 8). In an embodiment, the tumor homing peptide comprises a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In still another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence [AIB]VPI[AIB]QK (SEQ ID NO: 12) from the N-terminus of the apoptosis mediator Smac/DIABLO protein, wherein [AIB] is 2-Aminoisobutyric acid. In an embodiment, the branched reactive amino acid is a lysine, a cysteine, an azido-lysine or an azido-ornithine. In another embodiment, the branched reactive amino acid is cysteine. In some embodiments, the branched reactive amino acid is azido-lysine. In an embodiment, the lysine is L-lysine or D-lysine or an azido-lysine, i.e., 6-azido-L-lysine or 6-azido-D-lysine. In an embodiment, the reactive amino acid is a 5-azido-L-ornithine or 5-azido-d-ornithine. In some embodiments, the branched reactive amino acid is azido-lysine. In an embodiment, the tumor homing peptide comprises an active cellular targeting moiety (A) having a peptide sequence containing an RGD peptide motif consisting of the three amino acids Arg-Gly-Asp. In another embodiment, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2). In some embodiments, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4). In another embodiment, the cyclic peptide sequence containing the cyclic iRGD peptide motif is cyclized via a disulfide bond between cysteine at residue position 1 and cysteine at residue position 9. In some embodiments, the tumor homing peptide comprises an active cellular targeting moiety (A) having a peptide sequence containing a NGR motif consisting of the three amino acids Asn-Gly-Arg. In an embodiment, the tumor homing peptide comprises an active cellular targeting moiety (A) having a cyclic peptide sequence containing a NGR motif consisting of the five amino acids Cys-Asn-Gly-Arg-Cys (SEQ ID NO: 5). In an embodiment, the cyclic peptide sequence containing the NGR motif is cyclized with a disulfide bond between cysteine at residue position 1 and cysteine at residue position 5. In another embodiment, the tumor homing peptide comprises a cyclic peptide sequence consisting of the nine amino acid sequence Cys-Gly-Asn-Lys-Arg-Thr-Arg-Gly-Cys (SEQ ID NO: 6). In an embodiment, the cyclic peptide is cyclized with a disulfide bond between cysteine at residue position 1 and cysteine at residue position 9. In another embodiment, the tumor homing peptide comprises a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In some embodiments, the tumor homing peptide has a peptide sequence consisting of the amino acids Cys-Arg-Glu-Lys-Ala (SEQ ID NO: 8). In another embodiment, the tumor homing peptide comprises a peptide sequence consisting of the amino acids Cys-Arg-Glu-Lys-Ala (SEQ ID NO: 8). In an embodiment, the tumor homing peptide comprises a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In an embodiment of the herein described multifunctional therapeutic peptide conjugates comprises a tumor homing peptide, at least one protein binding moiety and a branched reactive amino acid, wherein the tumor homing peptide is conjugated to a first linker, the at least one protein binding moiety is conjugated to a second linker and the first and second linkers are conjugated to the branched reactive amino acid, wherein the at least one protein binding moiety is an inhibitor of apoptosis (IAP). In certain embodiments, the at least one protein binding moiety consists of a seven amino acid sequence AVPIAQK (SEQ ID NO: 10) from the N-terminus of the apoptosis mediator Smac/DIABLO protein. In some embodiments, the at least one protein binding moiety consists of a seven amino acid sequence 2-Aminoisobutyric acid [AIB]VPIAQK (SEQ ID NO: 11) from the N-terminus of the apoptosis mediator Smac/DIABLO protein. In an embodiment, the at least one protein binding moiety consists of a seven amino acid sequence [AIB]VPI[AIB]QK (SEQ ID NO: 12) from the N-terminus of the apoptosis mediator Smac/DIABLO protein. In a particular embodiment, the multifunctional therapeutic peptide conjugate comprises two protein binding moieties, wherein each protein binding moiety consists of a seven amino acid sequence AVPIAQK from the N-terminus of the apoptosis mediator Smac/DIABLO protein. In various embodiments, the multifunctional therapeutic peptide conjugate comprises two protein binding moieties, wherein each protein binding moiety consists of a seven amino acid sequence 2-Aminoisobutyric acid [AIB]VPIAQK (SEQ ID NO: 11) from the N-terminus of the apoptosis mediator Smac/DIABLO protein. In an embodiment, the multifunctional therapeutic peptide conjugate comprises two protein binding moieties, wherein each protein binding moiety consists of a seven amino acid sequence [AIB]VPI[AIB]QK (SEQ ID NO: 12) from the N-terminus of the apoptosis mediator Smac/DIABLO protein. In another embodiment, the multifunctional therapeutic peptide conjugate comprises three protein binding moieties, wherein each protein binding moiety consists of a seven amino acid sequence AVPIAQK (SEQ ID NO: 10) from the N-terminus of the apoptosis mediator Smac/DIABLO protein. In some embodiments, the multifunctional therapeutic peptide conjugate comprises three protein binding moieties, wherein each protein binding moiety consists of a seven amino acid sequence 2-Aminoisobutyric acid [AIB]VPIAQK (SEQ ID NO: 11) from the N-terminus of the apoptosis mediator Smac/DIABLO protein. In certain embodiments, the multifunctional therapeutic peptide conjugate comprises three protein binding moieties, wherein each protein binding moiety consists of a seven amino acid sequence [AIB]VPI[AIB]QK (SEQ ID NO: 12) from the N-terminus of the apoptosis mediator Smac/DIABLO protein.

In some embodiments of the herein provided multifunctional therapeutic peptide conjugate compounds, each of the first linker and the second linker is an identical linker. In certain embodiments of the herein provided multifunctional therapeutic peptide conjugate compounds each of the first linker and the second linker is a different linker. In some embodiments of the first linker and the second linker, the first linker and the second linker is polyethylene glycol (PEG). In an embodiment, wherein the first linker and the second linker is an identical polyethylene glycol (PEG) linker or a different PEG linker, the PEG is selected from the group consisting of amino-PEG2, amino-PEG3, amino-PEG4, amino-PEG6, amino-PEG8, amino-PEG10, amino-PEG12 and amino-PEG20.

In still another aspect, provided herein are multifunctional therapeutic peptide conjugates represented by formula (I):

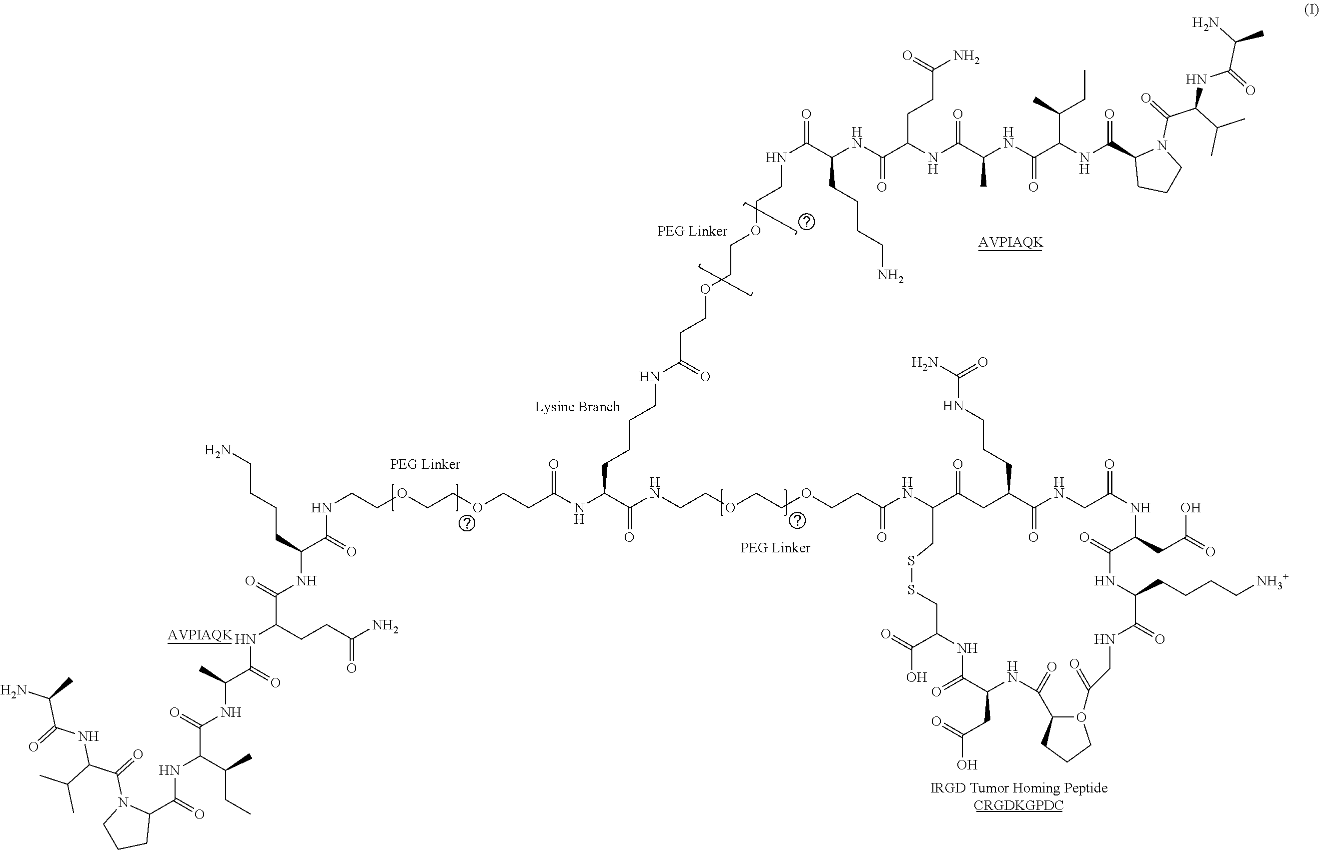

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence X₁X₂PIAQK (SEQ ID NO: 13) and X₁ is N-methyl alanine (MeA) and X₂ is tert-Leucine (Tle) (MeATlePIAQK (SEQ ID NO: 13)), from the N-terminus of the apoptosis mediator Smac/DIABLO protein.

In one aspect, provided herein are multifunctional therapeutic peptide conjugates represented by formula (II):

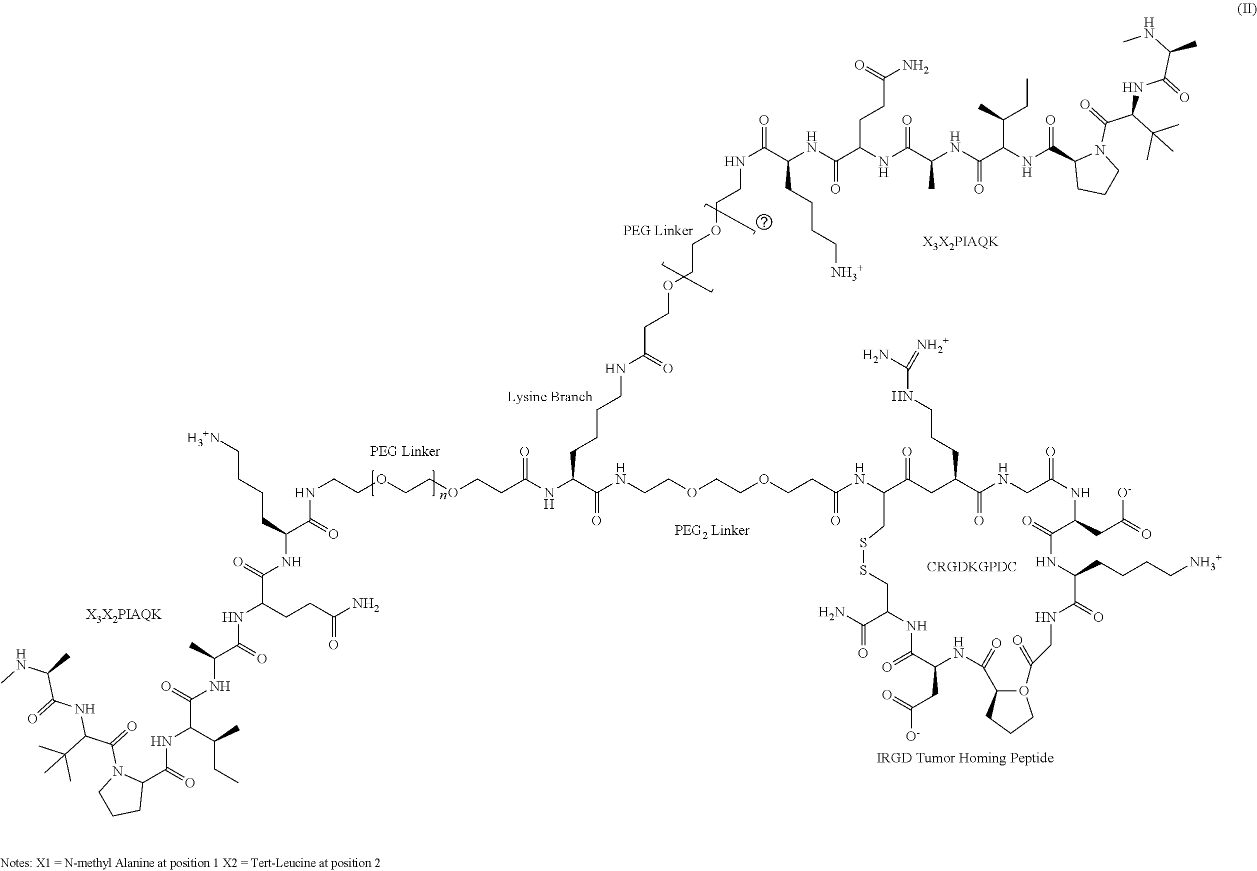

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein one of the two protein binding moiety consists of a seven amino acid sequence X₁X₂PFAQK (SEQ ID NO: 14), wherein X₁ is N-methyl alanine (MeA) and X₂ is tert-Leucine (Tle) (MeATlePFAQK (SEQ ID NO: 14)) and the second of the two protein binding moiety consists of a seven amino acid sequence X₁X₂PIAQK (SEQ ID NO: 13), wherein X₁ is N-methyl alanine (MeA) and X₂ is tert-Leucine (Tle) (MeATlePIAQK (SEQ ID NO: 13)), from the N-terminus of the apoptosis mediator Smac/DIABLO protein.

In one aspect, provided herein are multifunctional therapeutic peptide conjugates represented by formula (III):

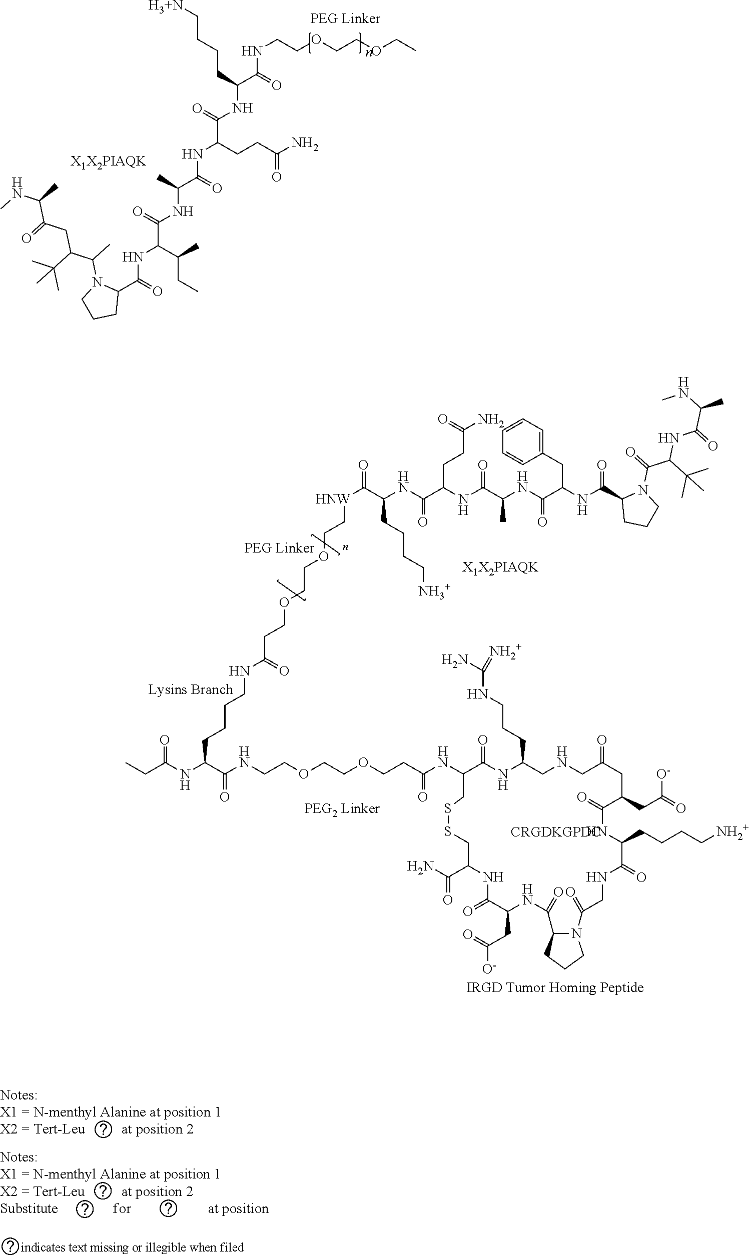

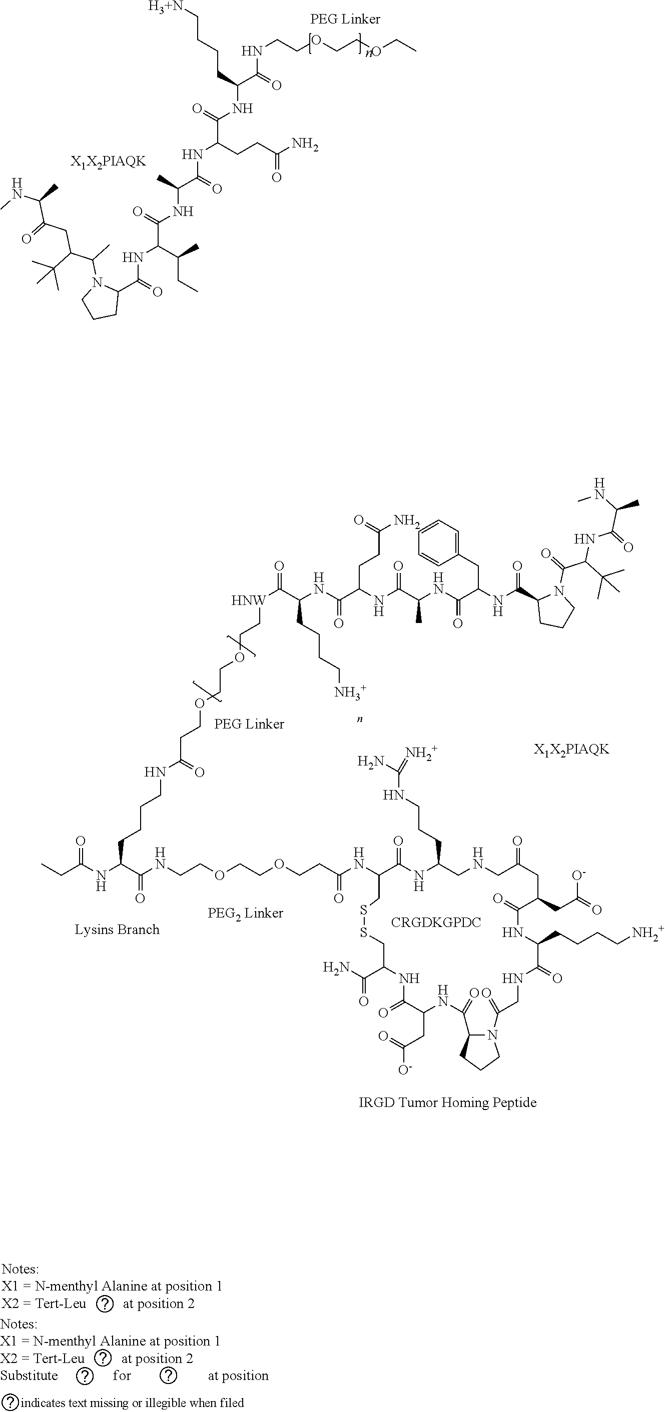

In another aspect, provided herein are methods for treating cancer comprising administering a to a subject in need thereof a therapeutically effective amount of a multifunctional therapeutic peptide conjugate compound to a subject in need thereof, the multifunctional therapeutic peptide conjugate compound comprising: a tumor homing peptide, a protein binding moiety and a branched reactive amino acid, wherein the tumor homing peptide is conjugated to a first linker, the protein binding moiety is conjugated to a second linker and the first and second linkers are conjugated to the branched reactive amino acid, wherein the protein binding moiety is an inhibitor of apoptosis (IAP).

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a peptide sequence containing an RGD peptide motif consisting of the three amino acids Arg-Gly-Asp.

In one aspect, provided herein are provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1).

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2).

In still another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3).

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4).

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a peptide sequence containing a NGR motif consisting of the three amino acids Asn-Gly-Arg.

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a NGR motif consisting of the five amino acids Cys-Asn-Gly-Arg-Cys (SEQ ID NO: 5).

In still another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein tumor homing peptide comprises a cyclic peptide sequence consisting of the nine amino acid sequence Cys-Gly-Asn-Lys-Arg-Thr-Arg-Gly-Cys (SEQ ID NO: 6).

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7).

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the amino acids Cys-Arg-Glu-Lys-Ala (SEQ ID NO: 8).

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise one protein binding moiety consisting of a seven amino acid sequence AVPIAQK (SEQ ID NO: 10) from the N-terminus of the apoptosis mediator Smac/DIABLO protein.

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence [AIB]VPIAQK (SEQ ID NO: 11) from the N-terminus of the apoptosis mediator Smac/DIABLO protein, wherein [AIB] is 2-Aminoisobutyric acid.

In still another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence [AIB]VPI[AIB]QK (SEQ ID NO: 12) from the N-terminus of the apoptosis mediator Smac/DIABLO protein, wherein [AIB] is 2-Aminoisobutyric acid.

In embodiments of the herein provided methods for treating cancer, the tumor homing peptide comprises an active cellular targeting moiety (A) having a peptide sequence containing an RGD peptide motif consisting of the three amino acids Arg-Gly-Asp. In another embodiment, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2). In some embodiments, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4). In another embodiment, the cyclic peptide sequence containing the cyclic iRGD peptide motif is cyclized via a disulfide bond between cysteine at residue position 1 and cysteine at residue position 9. In some embodiments, the tumor homing peptide comprises an active cellular targeting moiety (A) having a peptide sequence containing a NGR motif consisting of the three amino acids Asn-Gly-Arg. In an embodiment, the tumor homing peptide comprises an active cellular targeting moiety (A) having a cyclic peptide sequence containing a NGR motif consisting of the five amino acids Cys-Asn-Gly-Arg-Cys (SEQ ID NO: 5). In an embodiment, the cyclic peptide sequence containing the NGR motif is cyclized with a disulfide bond between cysteine at residue position 1 and cysteine at residue position 5. In another embodiment, the tumor homing peptide comprises a cyclic peptide sequence consisting of the nine amino acid sequence Cys-Gly-Asn-Lys-Arg-Thr-Arg-Gly-Cys (SEQ ID NO: 6). In an embodiment, the cyclic peptide is cyclized with a disulfide bond between cysteine at residue position 1 and cysteine at residue position 9. In another embodiment, the tumor homing peptide comprises a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In some embodiments, the tumor homing peptide comprises a peptide sequence consisting of the amino acids Cys-Arg-Glu-Lys-Ala (SEQ ID NO: 8). In another embodiment, the tumor homing peptide consists of the amino acids Cys-Arg-Glu-Lys-Ala. In an embodiment, the tumor homing peptide comprises a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In an embodiment of the herein described methods for treating cancer, the multifunctional therapeutic peptide conjugate comprises a tumor homing peptide, at least one protein binding moiety and a branched reactive amino acid, wherein the tumor homing peptide is conjugated to a first linker, the at least one protein binding moiety is conjugated to a second linker and the first and second linkers are conjugated to the branched reactive amino acid, wherein the at least one protein binding moiety is an inhibitor of apoptosis (IAP). In certain embodiments, the at least one protein binding moiety consists of a seven amino acid sequence AVPIAQK (SEQ ID NO: 10) from the N-terminus of the apoptosis mediator Smac/DIABLO protein. In some embodiments, the at least one protein binding moiety consists of a seven amino acid sequence 2-Aminoisobutyric acid [AIB]VPIAQK (SEQ ID NO: 11) from the N-terminus of the apoptosis mediator Smac/DIABLO protein. In an embodiment, the at least one protein binding moiety consists of a seven amino acid sequence [AIB]VPI[AIB]QK (SEQ ID NO: 12) from the N-terminus of the apoptosis mediator Smac/DIABLO protein. In a particular embodiment, the multifunctional therapeutic peptide conjugate comprises two protein binding moieties, wherein each protein binding moiety consists of a seven amino acid sequence AVPIAQK (SEQ ID NO: 10) from the N-terminus of the apoptosis mediator Smac/DIABLO protein. In various embodiments, the multifunctional therapeutic peptide conjugate comprises two protein binding moieties, wherein each protein binding moiety consists of a seven amino acid sequence 2-Aminoisobutyric acid [AIB]VPIAQK (SEQ ID NO: 11) from the N-terminus of the apoptosis mediator Smac/DIABLO protein. In an embodiment, the multifunctional therapeutic peptide conjugate comprises two protein binding moieties, wherein each protein binding moiety consists of a seven amino acid sequence [AIB]VPI[AIB]QK (SEQ ID NO: 12) from the N-terminus of the apoptosis mediator Smac/DIABLO protein. In another embodiment, the multifunctional therapeutic peptide conjugate comprises three protein binding moieties, wherein each protein binding moiety consists of a seven amino acid sequence AVPIAQK (SEQ ID NO: 10) from the N-terminus of the apoptosis mediator Smac/DIABLO protein. In some embodiments, the multifunctional therapeutic peptide conjugate comprises three protein binding moieties, wherein each protein binding moiety consists of a seven amino acid sequence 2-Aminoisobutyric acid [AIB]VPIAQK (SEQ ID NO: 11) from the N-terminus of the apoptosis mediator Smac/DIABLO protein. In certain embodiments, the multifunctional therapeutic peptide conjugate comprises three protein binding moieties, wherein each protein binding moiety consists of a seven amino acid sequence [AIB]VPI[AIB]QK (SEQ ID NO: 12) from the N-terminus of the apoptosis mediator Smac/DIABLO protein.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

Unless defined otherwise, all technical and scientific terms used herein have the same meaning as commonly understood to one of ordinary skill in the art.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject,” “individual,” and “patient” are used interchangeably herein, and refer to an animal, for example a human, to whom treatment for one or more cancer, including prophylactic treatment, with the pharmaceutical compositions according to the present invention, i.e., the herein described multifunctional branched therapeutic agents, respectively, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g., mouse or rat), guinea pig, goat, pig, cat, rabbits, cows, horses and non-mammals such as reptiles, amphibians, chickens, and turkeys.

### Tumor Targeting Proteins

In an embodiment of the multifunctional therapeutic peptide conjugate, the tumor homing peptide is the cyclic peptide iRGD having amino acid sequence CRGD[K/R]GP[D/E]C (SEQ ID NO: 1 and SEQ ID NO:4). In another embodiment, the tumor homing peptide is the cyclic peptide iRGD having amino acid sequence Cys-Asn-Gly-Arg-Cys (SEQ ID NO: 5). In another embodiment, the tumor homing peptide is the cyclic peptide iRGD having amino acid sequence Cys-Gly-Asn-Lys-Arg-Thr-Arg-Gly-Cys (SEQ ID NO: 6). In some embodiments, the tumor homing peptide is the cyclic peptide iRGD having amino acid sequence Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In an embodiment, the tumor homing peptide is the cyclic peptide iRGD having amino acid sequence Cys-Arg-Glu-Lys-Ala (SEQ ID NO: 8). In another embodiment, the tumor homing peptide is the cyclic peptide iRGD having amino acid sequence Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In some embodiments, the multifunctional therapeutic peptide conjugate comprises one protein binding moiety consisting of a seven amino acid sequence AVPIAQK (SEQ ID NO: 10) from the N-terminus of the apoptosis mediator Smac/DIABLO protein. In an embodiment, the multifunctional therapeutic peptide conjugate comprises one protein binding moiety consisting of a seven amino acid sequence [AIB]VPIAQK (SEQ ID NO: 11), wherein [AIB] is 2-Aminoisobutyric acid. In another embodiment, the multifunctional therapeutic peptide conjugate comprises one protein binding moiety consisting of a seven amino acid sequence [AIB]VPI[AIB]QK (SEQ ID NO: 12), wherein [AIB] is 2-Aminoisobutyric acid. In some embodiments, the multifunctional therapeutic peptide conjugate comprises two protein binding moieties, wherein each protein binding moiety consists of a seven amino acid sequence AVPIAQK (SEQ ID NO: 10) from the N-terminus of the apoptosis mediator Smac/DIABLO protein. In some embodiments, the first and second linker are the same. In certain embodiments, the first and second linker are different linker molecules.

As noted herein, the tumor-homing peptide known as iRGD protein. iRGD is cyclic peptide composed of 9-amino acids including an Arg-Gly-Asp (RGD) motif, has a high binding affinity to αvβ3 and αvβ5 integrins abundant in tumor vasculatures. IRGD protein has the following chemical structure:

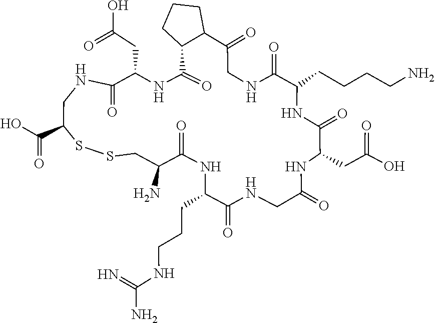

In some embodiments, the tumor-homing peptide compound is an iRGD protein analog or derivative thereof. In an embodiment, the iRGD protein analog or derivative thereof comprises RGD peptide motif consisting of the three amino acids Arg-Gly-Asp. In another embodiment, the iRGD protein analog or derivative thereof is RGD peptide motif consisting of the three amino acids Arg-Gly-Asp. In an embodiment, the iRGD protein analog or derivative thereof comprises iRGD motif consisting of the nine amino acid sequence Cys-Arg-Gly-Asp-Lys-Gly-Pro-Asp-Cys (SEQ ID NO: 1). In another embodiment, the iRGD protein analog or derivative thereof is iRGD motif consisting of the nine amino acid sequence Cys-Arg-Gly-Asp-Lys-Gly-Pro-Asp-Cys (SEQ ID NO: 1). In some embodiments, the iRGD protein analog or derivative thereof comprises the iRGD motif consisting of the nine amino acid sequence Cys-Arg-Gly-Asp-Arg-Gly-Pro-Asp-Cys (SEQ ID NO: 2). In some embodiments, the iRGD protein analog or derivative thereof is the iRGD motif consisting of the nine amino acid sequence Cys-Arg-Gly-Asp-Arg-Gly-Pro-Asp-Cys (SEQ ID NO: 2). In an embodiment, the iRGD protein analog or derivative thereof comprises the iRGD motif consisting of the nine amino acid sequence Cys-Arg-Gly-Asp-Lys-Gly-Pro-Glu-Cys (SEQ ID NO: 3). In another embodiment, the iRGD protein analog or derivative thereof is the iRGD motif consisting of the nine amino acid sequence Cys-Arg-Gly-Asp-Lys-Gly-Pro-Glu-Cys (SEQ ID NO: 3). In another embodiment, the iRGD protein analog or derivative thereof comprises the iRGD motif consisting of the nine amino acid sequence Cys-Arg-Gly-Asp-Arg-Gly-Pro-Glu-Cys (SEQ ID NO: 4). In another embodiment, the iRGD protein analog or derivative thereof is iRGD motif consisting of the nine amino acid sequence Cys-Arg-Gly-Asp-Arg-Gly-Pro-Glu-Cys (SEQ ID NO: 4).

In an embodiment, the tumor-homing peptide compound comprises the NGR motif consisting of the three amino acids Asn-Gly-Arg. In another embodiment, the tumor-homing peptide compound is the NGR motif consisting of the three amino acids Asn-Gly-Arg. In an embodiment, the tumor-homing peptide compound comprises an NGR motif consisting of the five amino acids Cys-Asn-Gly-Arg-Cys (SEQ ID NO: 5). In another embodiment, the tumor-homing peptide compound is an NGR motif consisting of the five amino acids Cys-Asn-Gly-Arg-Cys (SEQ ID NO: 5).

In an embodiment, the tumor-homing peptide compound comprises a peptide sequence containing the cyclic peptide consisting of the nine amino acid sequence Cys-Gly-Asn-Lys-Arg-Thr-Arg-Gly-Cys (SEQ ID NO: 6). In another embodiment, the tumor-homing peptide compound is a peptide sequence containing the cyclic peptide consisting of the nine amino acid sequence Cys-Gly-Asn-Lys-Arg-Thr-Arg-Gly-Cys (SEQ ID NO: 6).

In an embodiment, the tumor-homing peptide compound comprises a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In another embodiment, the tumor-homing peptide compound is an peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7).

In an embodiment, the tumor-homing peptide compound comprises a peptide sequence consisting of the five amino acids Cys-Arg-Glu-Lys-Ala (SEQ ID NO: 8). In another embodiment, the tumor-homing peptide compound is a peptide sequence consisting of the five amino acids Cys-Arg-Glu-Lys-Ala (SEQ ID NO: 8).

In an embodiment, the tumor-homing peptide compound comprises a peptide sequence consisting of the 21 amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9). In another embodiment, the tumor-homing peptide compound is a peptide sequence consisting of the 21 amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In an embodiment, the tumor-homing peptide compound comprises a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val-cysteamine (SEQ ID NO: 9 with a cystamine). In another embodiment, the tumor-homing peptide compound is a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val-cysteamine (SEQ ID NO: 9 with a cystamine).

Combinations of substituents envisioned by this invention are preferably those that result in the formation of stable or chemically feasible compounds. The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week. In each of the foregoing and each of the following embodiments, it is to be understood that the formulas also include any and all hydrates and/or solvates of the compound formulas. It is appreciated that certain functional groups, such as the hydroxy, amino, and like groups form complexes and/or coordination compounds with water and/or various solvents, in the various physical forms of the compounds. Accordingly, the above formulas are to be understood to include and represent those various hydrates and/or solvates.

As used herein, the term “solvate” refers to compounds that further include a stoichiometric or non-stoichiometric amount of a solvent bound by non-covalent intermolecular forces. If the solvent is water, the solvate is referred to as “hydrate.” Pharmaceutically acceptable solvates and hydrates are complexes that, for example, may include from 1 to about 100, or from 1 to about 10, or from one to about 2.3 or 4 molecules of water or a solvent. In some embodiments, the hydrate may be a channel hydrate. It should be understood that the term “compound” in this application covers the compound and solvates of the compound, as well as mixtures thereof.

In some embodiments, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes “pharmaceutically acceptable salts” of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the compound of the invention include the conventional non-toxic salts of the parent compound formed, for example, from non-toxic inorganic or organic acids. The pharmaceutically acceptable salts of the compound of the invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. In some embodiments, the solvent is a nonaqueous media like ether, ethyl acetate, ethanol, isopropanol, or acetonitrile.

The pharmaceutically acceptable salts of the compound of the invention can be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of an existing salt for another ion or suitable ion-exchange resin.

Possible pharmaceutically acceptable salts are well known in the art. For example, S. M. Berge, et al. describe pharmaceutically acceptable salts in detail in J. Pharmaceutical Sciences, 66:1-19, 1977; which is incorporated herein by reference in its entirety.

The invention further includes analogs of the compound of the invention. The term “derivatives” includes but is not limited to ether derivatives, acid derivatives, amide derivatives, ester derivatives and the like.

The invention further includes pharmaceutical products of the compound of the invention. The term “pharmaceutical product” means a composition suitable for pharmaceutical use (pharmaceutical composition), as defined herein.

### Multifunctional Therapeutic Agent

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the protein binding moiety is an inhibitor of apoptosis (IAP).

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a peptide sequence containing an RGD peptide motif consisting of the three amino acids Arg-Gly-Asp.

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1).

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2).

In still another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3).

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4).

In embodiments of the above-described multifunctional therapeutic peptide conjugate compounds comprising an active cellular targeting moiety (A) having a cyclic peptide sequence, the cyclic peptide sequence containing the cyclic iRGD peptide motif is cyclized via a disulfide bond between cysteine at residue position 1 and cysteine at residue position 9.

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a peptide sequence containing a NGR motif consisting of the three amino acids Asn-Gly-Arg.

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a NGR motif consisting of the five amino acids Cys-Asn-Gly-Arg-Cys (SEQ ID NO: 5).

In certain embodiments of the above-described multifunctional therapeutic peptide conjugate compounds comprising an active cellular targeting moiety (A) having a cyclic peptide sequence containing the NGR motif, the cyclic peptide sequence is cyclized with a disulfide bond between cysteine at residue position 1 and cysteine at residue position 5. In an embodiment of the above-described multifunctional therapeutic peptide conjugate compounds comprising an active cellular targeting moiety (A) having a cyclic peptide sequence containing the NGR motif, the active cellular targeting moiety targets the extracellular enzyme Aminopeptidase N (CD13).

In still another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein tumor homing peptide comprises a cyclic peptide sequence consisting of the nine amino acid sequence Cys-Gly-Asn-Lys-Arg-Thr-Arg-Gly-Cys (SEQ ID NO: 6).

In an embodiment of the above-described multifunctional therapeutic peptide conjugate compounds comprising the cyclic peptide sequence consisting of the nine amino acid sequence Cys-Gly-Asn-Lys-Arg-Thr-Arg-Gly-Cys (SEQ ID NO: 6), the cyclic peptide is cyclized with a disulfide bond between cysteine at residue position 1 and cysteine at residue position 9. In some embodiments of said multifunctional therapeutic peptide conjugate compounds, the active cellular targeting moiety targets the cellular receptor p32/gC1qR.

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In an embodiment of said multifunctional therapeutic peptide conjugate compounds, the active cellular targeting moiety targets the Nucleolin cellular receptor.

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the amino acids Cys-Arg-Glu-Lys-Ala (SEQ ID NO: 8). In an embodiment of said multifunctional therapeutic peptide conjugate compounds, the active cellular targeting moiety targets Fibrin-fibronectin complexes.

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9). In an embodiment of said multifunctional therapeutic peptide conjugate compounds, the active cellular targeting moiety targets the cellular receptor GRP78.

In an embodiment of the above-described multifunctional therapeutic peptide conjugate compounds, the branched reactive amino acid is a lysine, a cysteine, an azido-lysine or an azido-ornithine. In a particular embodiment, the branched reactive amino acid is lysine. In another embodiment, the branched reactive amino acid is cysteine. In some embodiments, the branched reactive amino acid is azido-lysine. In an embodiment, the lysine is L-lysine or D-lysine or an azido-lysine, i.e., 6-azido-L-lysine or 6-azido-D-lysine. In an embodiment, the reactive amino acid is a 5-azido-L-ornithine or 5-azido-d-ornithine.

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise one protein binding moiety consisting of a seven amino acid sequence AVPIAQK (SEQ ID NO: 10) from the N-terminus of the apoptosis mediator Smac/DIABLO protein. In an embodiment, the branched reactive amino acid is a lysine, a cysteine, an azido-lysine or an azido-ornithine. In a particular embodiment, the branched reactive amino acid is lysine. In another embodiment, the branched reactive amino acid is cysteine. In some embodiments, the branched reactive amino acid is azido-lysine. In an embodiment, the lysine is L-lysine or D-lysine or an azido-lysine, i.e., 6-azido-L-lysine or 6-azido-D-lysine. In an embodiment, the reactive amino acid is a 5-azido-L-ornithine or 5-azido-d-ornithine. In an embodiment, the tumor homing peptide comprises an active cellular targeting moiety (A) having a peptide sequence containing an RGD peptide motif consisting of the three amino acids Arg-Gly-Asp. In another embodiment, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2). In some embodiments, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4). In another embodiment, the cyclic peptide sequence containing the cyclic iRGD peptide motif is cyclized via a disulfide bond between cysteine at residue position 1 and cysteine at residue position 9. In some embodiments, the tumor homing peptide comprises an active cellular targeting moiety (A) having a peptide sequence containing a NGR motif consisting of the three amino acids Asn-Gly-Arg. In an embodiment, the tumor homing peptide comprises an active cellular targeting moiety (A) having a cyclic peptide sequence containing a NGR motif consisting of the five amino acids Cys-Asn-Gly-Arg-Cys (SEQ ID NO: 5). In an embodiment, the cyclic peptide sequence containing the NGR motif is cyclized with a disulfide bond between cysteine at residue position 1 and cysteine at residue position 5. In another embodiment, the tumor homing peptide comprises a cyclic peptide sequence consisting of the nine amino acid sequence Cys-Gly-Asn-Lys-Arg-Thr-Arg-Gly-Cys (SEQ ID NO: 6). In an embodiment, the cyclic peptide is cyclized with a disulfide bond between cysteine at residue position 1 and cysteine at residue position 9. In another embodiment, the tumor homing peptide comprises a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In some embodiments, the tumor homing peptide has a peptide sequence consisting of the amino acids Cys-Arg-Glu-Lys-Ala (SEQ ID NO: 8). In another embodiment, the tumor homing peptide comprises a peptide sequence consisting of the amino acids Cys-Arg-Glu-Lys-Ala (SEQ ID NO: 8). In an embodiment, the tumor homing peptide comprises a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In one aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise one protein binding moiety consisting of a seven amino acid sequence consists of a seven amino acid sequence [AIB]VPIAQK (SEQ ID NO: 11) from the N-terminus of the apoptosis mediator Smac/DIABLO protein, wherein [AIB] is 2-Aminoisobutyric acid. In an embodiment, the branched reactive amino acid is a lysine, a cysteine, an azido-lysine or an azido-ornithine. In a particular embodiment, the branched reactive amino acid is lysine. In another embodiment, the branched reactive amino acid is cysteine. In some embodiments, the branched reactive amino acid is azido-lysine. In an embodiment, the lysine is L-lysine or D-lysine or an azido-lysine, i.e., 6-azido-L-lysine or 6-azido-D-lysine. In an embodiment, the reactive amino acid is a 5-azido-L-ornithine or 5-azido-d-ornithine. In an embodiment, the tumor homing peptide comprises an active cellular targeting moiety (A) having a peptide sequence containing an RGD peptide motif consisting of the three amino acids Arg-Gly-Asp. In another embodiment, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2). In some embodiments, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4). In another embodiment, the cyclic peptide sequence containing the cyclic iRGD peptide motif is cyclized via a disulfide bond between cysteine at residue position 1 and cysteine at residue position 9. In some embodiments, the tumor homing peptide comprises an active cellular targeting moiety (A) having a peptide sequence containing a NGR motif consisting of the three amino acids Asn-Gly-Arg. In an embodiment, the tumor homing peptide comprises an active cellular targeting moiety (A) having a cyclic peptide sequence containing a NGR motif consisting of the five amino acids Cys-Asn-Gly-Arg-Cys (SEQ ID NO: 5). In an embodiment, the cyclic peptide sequence containing the NGR motif is cyclized with a disulfide bond between cysteine at residue position 1 and cysteine at residue position 5. In another embodiment, the tumor homing peptide comprises a cyclic peptide sequence consisting of the nine amino acid sequence Cys-Gly-Asn-Lys-Arg-Thr-Arg-Gly-Cys (SEQ ID NO: 6). In an embodiment, the cyclic peptide is cyclized with a disulfide bond between cysteine at residue position 1 and cysteine at residue position 9. In another embodiment, the tumor homing peptide comprises a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In some embodiments, the tumor homing peptide has a peptide sequence consisting of the amino acids Cys-Arg-Glu-Lys-Ala (SEQ ID NO: 8). In another embodiment, the tumor homing peptide comprises a peptide sequence consisting of the amino acids Cys-Arg-Glu-Lys-Ala (SEQ ID NO: 8). In an embodiment, the tumor homing peptide comprises a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9). In an embodiment, the tumor homing peptide comprises an active cellular targeting moiety (A) having a peptide sequence containing an RGD peptide motif consisting of the three amino acids Arg-Gly-Asp. In another embodiment, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2). In some embodiments, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4). In another embodiment, the cyclic peptide sequence containing the cyclic iRGD peptide motif is cyclized via a disulfide bond between cysteine at residue position 1 and cysteine at residue position 9. In some embodiments, the tumor homing peptide comprises an active cellular targeting moiety (A) having a peptide sequence containing a NGR motif consisting of the three amino acids Asn-Gly-Arg. In an embodiment, the tumor homing peptide comprises an active cellular targeting moiety (A) having a cyclic peptide sequence containing a NGR motif consisting of the five amino acids Cys-Asn-Gly-Arg-Cys (SEQ ID NO: 5). In an embodiment, the cyclic peptide sequence containing the NGR motif is cyclized with a disulfide bond between cysteine at residue position 1 and cysteine at residue position 5. In another embodiment, the tumor homing peptide comprises a cyclic peptide sequence consisting of the nine amino acid sequence Cys-Gly-Asn-Lys-Arg-Thr-Arg-Gly-Cys (SEQ ID NO: 6). In an embodiment, the cyclic peptide is cyclized with a disulfide bond between cysteine at residue position 1 and cysteine at residue position 9. In another embodiment, the tumor homing peptide comprises a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In some embodiments, the tumor homing peptide has a peptide sequence consisting of the amino acids Cys-Arg-Glu-Lys-Ala (SEQ ID NO: 8). In another embodiment, the tumor homing peptide comprises a peptide sequence consisting of the amino acids Cys-Arg-Glu-Lys-Ala. In an embodiment, the tumor homing peptide comprises a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise one protein binding moiety consisting of a seven amino acid sequence [AIB]VPI[AIB]QK (SEQ ID NO: 11) from the N-terminus of the apoptosis mediator Smac/DIABLO protein, wherein [AIB] is 2-Aminoisobutyric acid. In an embodiment, the branched reactive amino acid is a lysine, a cysteine, an azido-lysine or an azido-ornithine. In a particular embodiment, the branched reactive amino acid is lysine. In another embodiment, the branched reactive amino acid is cysteine. In some embodiments, the branched reactive amino acid is azido-lysine. In some embodiments, the branched reactive amino acid is azido-lysine. In an embodiment, the lysine is L-lysine or D-lysine or an azido-lysine, i.e., 6-azido-L-lysine or 6-azido-D-lysine. In an embodiment, the reactive amino acid is a 5-azido-L-ornithine or 5-azido-d-ornithine. In an embodiment, the tumor homing peptide comprises an active cellular targeting moiety (A) having a peptide sequence containing an RGD peptide motif consisting of the three amino acids Arg-Gly-Asp. In another embodiment, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2). In some embodiments, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4). In another embodiment, the cyclic peptide sequence containing the cyclic iRGD peptide motif is cyclized via a disulfide bond between cysteine at residue position 1 and cysteine at residue position 9. In some embodiments, the tumor homing peptide comprises an active cellular targeting moiety (A) having a peptide sequence containing a NGR motif consisting of the three amino acids Asn-Gly-Arg. In an embodiment, the tumor homing peptide comprises an active cellular targeting moiety (A) having a cyclic peptide sequence containing a NGR motif consisting of the five amino acids Cys-Asn-Gly-Arg-Cys (SEQ ID NO: 5). In an embodiment, the cyclic peptide sequence containing the NGR motif is cyclized with a disulfide bond between cysteine at residue position 1 and cysteine at residue position 5. In another embodiment, the tumor homing peptide comprises a cyclic peptide sequence consisting of the nine amino acid sequence Cys-Gly-Asn-Lys-Arg-Thr-Arg-Gly-Cys (SEQ ID NO: 6). In an embodiment, the cyclic peptide is cyclized with a disulfide bond between cysteine at residue position 1 and cysteine at residue position 9. In another embodiment, the tumor homing peptide comprises a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In some embodiments, the tumor homing peptide has a peptide sequence consisting of the amino acids Cys-Arg-Glu-Lys-Ala (SEQ ID NO: 8). In another embodiment, the tumor homing peptide comprises a peptide sequence consisting of the amino acids Cys-Arg-Glu-Lys-Ala. In an embodiment, the tumor homing peptide comprises a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 3).

In another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence [AIB]VPIAQK (SEQ ID NO: 11) from the N-terminus of the apoptosis mediator Smac/DIABLO protein, wherein [AIB] is 2-Aminoisobutyric acid. In an embodiment, the branched reactive amino acid is a lysine, a cysteine, an azido-lysine or an azido-ornithine. In a particular embodiment, the branched reactive amino acid is lysine. In another embodiment, the branched reactive amino acid is cysteine. In some embodiments, the branched reactive amino acid is azido-lysine. In an embodiment, the lysine is L-lysine or D-lysine or an azido-lysine, i.e., 6-azido-L-lysine or 6-azido-D-lysine. In an embodiment, the reactive amino acid is a 5-azido-L-ornithine or 5-azido-d-ornithine. In an embodiment, the branched reactive amino acid is lysine, cysteine or azido-lysine. In a particular embodiment, the branched reactive amino acid is lysine. In another embodiment, the branched reactive amino acid is cysteine. In some embodiments, the branched reactive amino acid is azido-lysine. In an embodiment, the lysine is L-lysine or D-lysine or an azido-lysine, i.e., 6-azido-L-lysine or 6-azido-D-lysine. In an embodiment, the reactive amino acid is a 5-azido-L-ornithine or 5-azido-d-ornithine. In an embodiment, the tumor homing peptide comprises an active cellular targeting moiety (A) having a peptide sequence containing an RGD peptide motif consisting of the three amino acids Arg-Gly-Asp. In another embodiment, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2). In some embodiments, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4). In another embodiment, the cyclic peptide sequence containing the cyclic iRGD peptide motif is cyclized via a disulfide bond between cysteine at residue position 1 and cysteine at residue position 9. In some embodiments, the tumor homing peptide comprises an active cellular targeting moiety (A) having a peptide sequence containing a NGR motif consisting of the three amino acids Asn-Gly-Arg. In an embodiment, the tumor homing peptide comprises an active cellular targeting moiety (A) having a cyclic peptide sequence containing a NGR motif consisting of the five amino acids Cys-Asn-Gly-Arg-Cys (SEQ ID NO: 5). In an embodiment, the cyclic peptide sequence containing the NGR motif is cyclized with a disulfide bond between cysteine at residue position 1 and cysteine at residue position 5. In another embodiment, the tumor homing peptide comprises a cyclic peptide sequence consisting of the nine amino acid sequence Cys-Gly-Asn-Lys-Arg-Thr-Arg-Gly-Cys (SEQ ID NO: 6). In an embodiment, the cyclic peptide is cyclized with a disulfide bond between cysteine at residue position 1 and cysteine at residue position 9. In another embodiment, the tumor homing peptide comprises a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In some embodiments, the tumor homing peptide has a peptide sequence consisting of the amino acids Cys-Arg-Glu-Lys-Ala. In another embodiment, the tumor homing peptide comprises a peptide sequence consisting of the amino acids Cys-Arg-Glu-Lys-Ala (SEQ ID NO: 8). In an embodiment, the tumor homing peptide comprises a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In still another aspect, provided herein are multifunctional therapeutic peptide conjugate compounds comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence [AIB]VPI[AIB]QK (SEQ ID NO: 12) from the N-terminus of the apoptosis mediator Smac/DIABLO protein, wherein [AIB] is 2-Aminoisobutyric acid. In an embodiment, the branched reactive amino acid is a lysine, a cysteine, an azido-lysine or an azido-ornithine. In a particular embodiment, the branched reactive amino acid is lysine. In another embodiment, the branched reactive amino acid is cysteine. In some embodiments, the branched reactive amino acid is azido-lysine. In an embodiment, the lysine is L-lysine or D-lysine or an azido-lysine, i.e., 6-azido-L-lysine or 6-azido-D-lysine. In an embodiment, the reactive amino acid is a 5-azido-L-ornithine or 5-azido-d-ornithine. In an embodiment, the tumor homing peptide comprises an active cellular targeting moiety (A) having a peptide sequence containing an RGD peptide motif consisting of the three amino acids Arg-Gly-Asp. In another embodiment, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2). In some embodiments, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4). In another embodiment, the cyclic peptide sequence containing the cyclic iRGD peptide motif is cyclized via a disulfide bond between cysteine at residue position 1 and cysteine at residue position 9. In some embodiments, the tumor homing peptide comprises an active cellular targeting moiety (A) having a peptide sequence containing a NGR motif consisting of the three amino acids Asn-Gly-Arg. In an embodiment, the tumor homing peptide comprises an active cellular targeting moiety (A) having a cyclic peptide sequence containing a NGR motif consisting of the five amino acids Cys-Asn-Gly-Arg-Cys (SEQ ID NO: 5). In an embodiment, the cyclic peptide sequence containing the NGR motif is cyclized with a disulfide bond between cysteine at residue position 1 and cysteine at residue position 5. In another embodiment, the tumor homing peptide comprises a cyclic peptide sequence consisting of the nine amino acid sequence Cys-Gly-Asn-Lys-Arg-Thr-Arg-Gly-Cys (SEQ ID NO: 6). In an embodiment, the cyclic peptide is cyclized with a disulfide bond between cysteine at residue position 1 and cysteine at residue position 9. In another embodiment, the tumor homing peptide comprises a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In some embodiments, the tumor homing peptide has a peptide sequence consisting of the amino acids Cys-Arg-Glu-Lys-Ala (SEQ ID NO: 8). In another embodiment, the tumor homing peptide comprises a peptide sequence consisting of the amino acids Cys-Arg-Glu-Lys-Ala (SEQ ID NO: 8). In an embodiment, the tumor homing peptide comprises a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In one aspect, the present the invention provides a multifunctional branched therapeutic agent represented by formula (I):

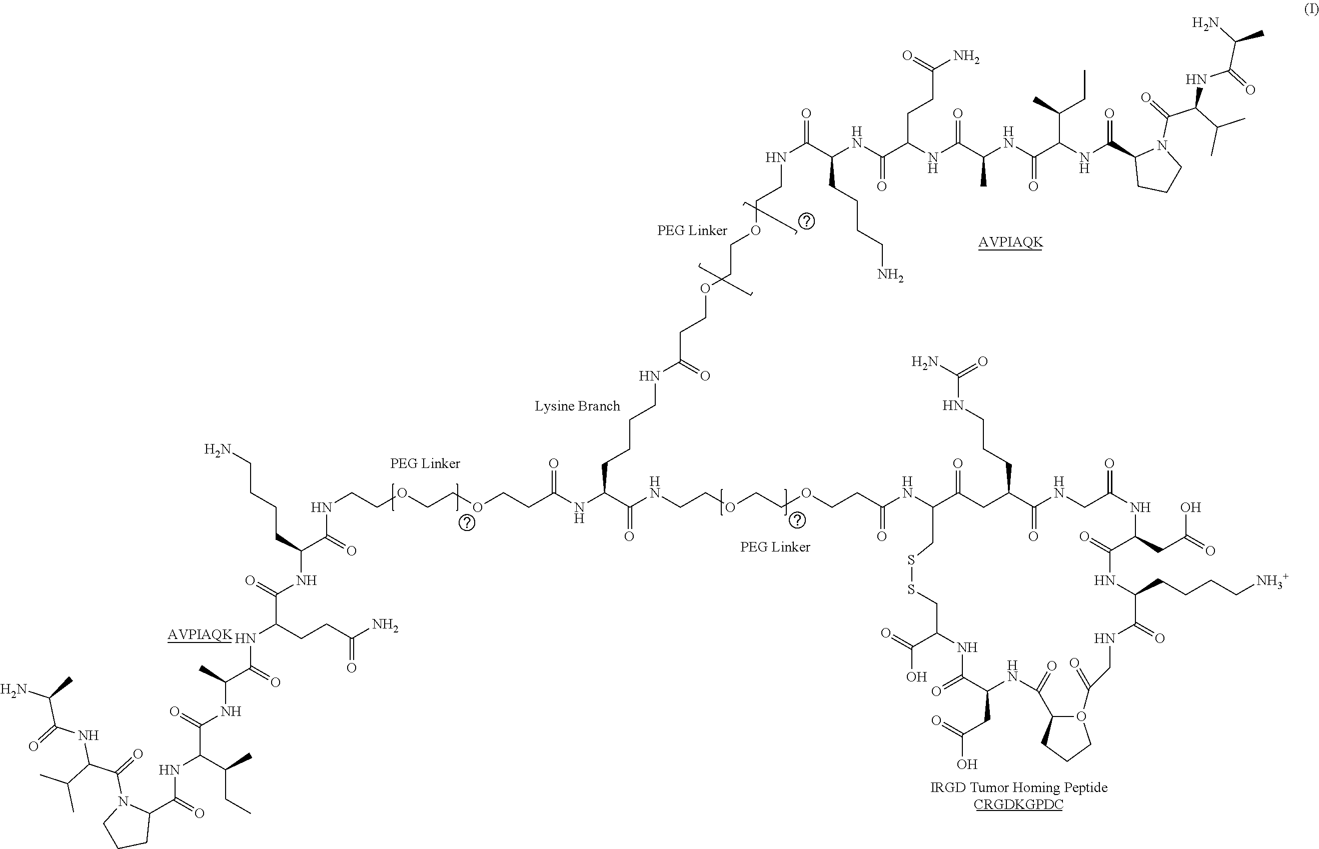

In an embodiment of the herein provided multifunctional therapeutic peptide conjugate compounds, each of the first linker and the second linker is an identical linker. In certain embodiments of the herein provided multifunctional therapeutic peptide conjugate compounds each of the first linker and the second linker is a different linker. In some embodiments of the of the first linker and the second linker, the first linker and the second linker is polyethylene glycol (PEG). In an embodiment, wherein the first linker and the second linker is an identical polyethylene glycol (PEG) linker or a different PEG linker, the PEG is selected from the group consisting of amino-PEG2, amino-PEG3, amino-PEG4, amino-PEG6, amino-PEG8, amino-PEG10, amino-PEG12 and amino-PEG20.

### Therapeutic Methods

In one aspect, the present the invention provides a method for treating cancer comprising administering to a subject in need thereof a therapeutically effective amount of any of the bifunctional and/or multifunctional therapeutic peptide conjugates described herein.

In another aspect, provided herein are methods for treating cancer comprising administering a to a subject in need thereof a therapeutically effective amount of a multifunctional therapeutic peptide conjugate compound to a subject in need thereof, the multifunctional therapeutic peptide conjugate compound comprising: a tumor homing peptide, a protein binding moiety and a branched reactive amino acid, wherein the tumor homing peptide is conjugated to a first linker, the protein binding moiety is conjugated to a second linker and the first and second linkers are conjugated to the branched reactive amino acid, wherein the protein binding moiety is an inhibitor of apoptosis (IAP).

In a particular aspect, the present the invention provides a method for treating cancer comprising administering a to a subject in need thereof a therapeutically effective amount of an improved bifunctional therapeutic peptide conjugate comprising the N-terminus of the Smac/Diablo protein consisting of seven amino acid sequence AVPIAQK (SEQ ID NO: 10) and the iRGD peptide, wherein the N-terminus of the Smac/Diablo protein and the iRGD peptide are connected to a branched reactive amino acid by a molecular linker. In an embodiment of the provided methods, the branched reactive amino acid is lysine. In an embodiment, the molecular linker is polyethylene glycol (PEG).

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a peptide sequence containing an RGD peptide motif consisting of the three amino acids Arg-Gly-Asp.

In one aspect, provided herein are provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1).

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2).

In still another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3).

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4).

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a peptide sequence containing a NGR motif consisting of the three amino acids Asn-Gly-Arg.

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide comprises an active cellular targeting moiety (A) having a cyclic peptide sequence containing a NGR motif consisting of the five amino acids Cys-Asn-Gly-Arg-Cys (SEQ ID NO: 5).

In still another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein tumor homing peptide comprises a cyclic peptide sequence consisting of the nine amino acid sequence Cys-Gly-Asn-Lys-Arg-Thr-Arg-Gly-Cys (SEQ ID NO: 6).

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7).

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the amino acids Cys-Arg-Glu-Lys-Ala (SEQ ID NO: 8).

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the tumor homing peptide has a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise one protein binding moiety consisting of a seven amino acid sequence AVPIAQK (SEQ ID NO: 10) from the N-terminus of the apoptosis mediator Smac/DIABLO protein.

In another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence [AIB]VPIAQK (SEQ ID NO: 11) from the N-terminus of the apoptosis mediator Smac/DIABLO protein, wherein [AIB] is 2-Aminoisobutyric acid.

In still another aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence [AIB]VPI[AIB]QK (SEQ ID NO: 12) from the N-terminus of the apoptosis mediator Smac/DIABLO protein, wherein [AIB] is 2-Aminoisobutyric acid.

In another aspect, provided herein are methods for treating cancer comprising administering a to a subject in need thereof a therapeutically effective amount of a multifunctional therapeutic peptide conjugate represented by formula (I), as described herein.

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein each protein binding moiety consists of a seven amino acid sequence X₁X₂PIAQK (SEQ ID NO: 13) and X₁ is N-methylated alanine (MeA) and X₂ is tert-Leucine (Tle) (i.e., MeATlePIAQK (SEQ ID NO: 13)), from the N-terminus of the apoptosis mediator Smac/DIABLO protein.

In another aspect, provided herein are methods for treating cancer comprising administering a to a subject in need thereof a therapeutically effective amount of a multifunctional therapeutic peptide conjugate represented by formula (II), as described herein.

In one aspect, provided herein are methods for treating cancer comprising administering to a subject in need thereof a therapeutically effective amount of a multifunctional therapeutic peptide conjugate compound comprising a tumor homing peptide, a protein binding moiety and a branched reactive amino acid, wherein the tumor homing peptide is conjugated to a first linker, the protein binding moiety is conjugated to a second linker, and the first and second linkers are conjugated to the branched reactive amino acid, wherein the compounds comprise two protein binding moieties, wherein one of the two protein binding moiety consists of a seven amino acid sequence X₁X₂PFAQK (SEQ ID NO: 14) (i.e., MeATlePFAQK (SEQ ID NO: 14)), wherein X₁ is N-methylated alanine (MeA) and X₂ is tert-Leucine (Tle) and the second of the two protein binding moiety consists of a seven amino acid sequence X₁X₂PIAQK (SEQ ID NO: 13) (i.e., MeATlePIAQK (SEQ ID NO: 13)), from the N-terminus of the apoptosis mediator Smac/DIABLO protein.

In another aspect, provided herein are methods for treating cancer comprising administering a to a subject in need thereof a therapeutically effective amount of a multifunctional therapeutic peptide conjugate represented by formula (III), as described herein.

In embodiments of the herein provided methods for treating cancer, the tumor homing peptide comprises an active cellular targeting moiety (A) having a peptide sequence containing an RGD peptide motif consisting of the three amino acids Arg-Gly-Asp. In another embodiment, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Asp-Cys (SEQ ID NO: 1). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Asp-Cys (SEQ ID NO: 2). In some embodiments, the tumor homing peptide comprises an active cellular targeting moiety (A) having a cyclic peptide sequence containing a cyclic iRGD peptide motif consisting of the nine amino acids Cys-Arg-Gly-Asp-Lys-Gly-Pro-Glu-Cys (SEQ ID NO: 3). In an embodiment, the tumor homing peptide comprises an active cellular targeting moiety (A) having a cyclic peptide sequence containing a cyclic iRGD peptide motif consisting of the nine amino acids Cys-Arg-Gly-Asp-Arg-Gly-Pro-Glu-Cys (SEQ ID NO: 4). In another embodiment, the cyclic peptide sequence containing the cyclic iRGD peptide motif is cyclized via a disulfide bond between cysteine at residue position 1 and cysteine at residue position 9. In some embodiments, the tumor homing peptide comprises an active cellular targeting moiety (A) having a peptide sequence containing a NGR motif consisting of the three amino acids Asn-Gly-Arg. In an embodiment, the tumor homing peptide comprises an active cellular targeting moiety (A) having a cyclic peptide sequence containing a NGR motif consisting of the five amino acids Cys-Asn-Gly-Arg-Cys (SEQ ID NO: 5). In an embodiment, the cyclic peptide sequence containing the NGR motif is cyclized with a disulfide bond between cysteine at residue position 1 and cysteine at residue position 5. In another embodiment, the tumor homing peptide comprises a cyclic peptide sequence consisting of the nine amino acid sequence Cys-Gly-Asn-Lys-Arg-Thr-Arg-Gly-Cys (SEQ ID NO: 6). In an embodiment, the cyclic peptide is cyclized with a disulfide bond between cysteine at residue position 1 and cysteine at residue position 9. In another embodiment, the tumor homing peptide comprises a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7). In some embodiments, the tumor homing peptide comprises a peptide sequence consisting of the amino acids Cys-Arg-Glu-Lys-Ala (SEQ ID NO: 8). In another embodiment, the tumor homing peptide consists of the amino acids Cys-Arg-Glu-Lys-Ala (SEQ ID NO: 8). In an embodiment, the tumor homing peptide comprises a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

In another aspect, the present the invention provides a method for treating cancer comprising administering a to a subject in need thereof a therapeutically effective amount of an multifunctional therapeutic peptide conjugate comprises a tumor homing peptide, a protein binding moiety and a branched reactive amino acid, wherein the tumor homing peptide is conjugated to a first linker, the protein binding moiety is conjugated to a second linker and the first and second linkers are conjugated to the branched reactive amino acid, wherein the protein binding moiety is an inhibitor of apoptosis (IAP) described herein comprising the above-described compounds.

In an embodiment of the herein provided method for treating cancer, the cancer is lung cancer, liver cancer, bladder cancer, breast cancer, colorectal cancer, esophageal cancer, gastric cancer, ovarian cancer, pancreatic cancer, or any other solid or hematological tumor.

It is one aspect of the present invention that the therapeutic compounds of the invention are used for treatment of cancer.

In some embodiments, the cancer is lung cancer, liver cancer, bladder cancer, breast cancer, colorectal cancer, esophageal cancer, gastric cancer, ovarian cancer, pancreatic cancer, or any other solid or hematological tumor. In some embodiments, the cancer is lung cancer. In some embodiments, the cancer is liver cancer. In some embodiments, the cancer is bladder cancer. In some embodiments, the cancer is breast cancer. In some embodiments, the cancer is colorectal cancer. In some embodiments, the cancer is esophageal cancer. In some embodiments, the cancer is gastric cancer. In some embodiments, the cancer is ovarian cancer. In some embodiments, the cancer is pancreatic cancer. In some embodiments, the cancer is a solid tumor. In some embodiments, the cancer is a hematological tumor. In some embodiments, the cancer is bile duct cancer. In other embodiments, the cancer is rhabdomyosarcoma. In some embodiments, the cancer is pediatric rhabdomyosarcoma. In some embodiments, the cancer is non-small cell lung cancer. In one embodiment, the compound of the invention suppresses the growth of cancer cells in vitro or in vivo.

The present invention further provides a method for treating a cancer in a subject in need thereof comprising administering to the subject a therapeutically effective amount of a compound, i.e., bifunctional or multifunctional therapeutic agent of the invention as described herein, or a pharmaceutically acceptable salt thereof. In another aspect, the present invention provides a pharmaceutical composition comprising a therapeutically effective amount of a compound of the invention, or a pharmaceutically acceptable salt thereof, and a pharmaceutically acceptable carrier.

A “therapeutically effective amount” as used herein refers to that amount which provides a therapeutic effect for a given indication and administration regimen.

In some embodiments, the cancer that can be treated by the compound of the invention includes but is not limited to cancerous and precancerous conditions, including, for example, premalignant and malignant hyperproliferative diseases. In another embodiment, the compounds of the invention are used to prevent and/or treat premalignant conditions and to prevent progression to a neoplastic or malignant state, including but not limited to those disorders described above. Such uses are indicated in conditions known to precede or suspected of preceding progression to neoplasia or cancer.

As used herein, a “pharmaceutically acceptable carrier” is well known to those skilled in the art. The carrier may be a solid carrier for solid formulations, a liquid carrier or diluent for liquid formulations, or mixtures thereof. In addition, the pharmaceutical compositions of the invention may further include one or more ingredient selected from diluents, buffers, flavoring agents, binders, disintegrants, surface active agents, thickeners, lubricants, preservatives (including antioxidants), and the like. The formulations may be of immediate release, sustained release, delayed-onset release or any other release profile known to one skilled in the art.

The compound, i.e., therapeutic agent, of the invention as described herein and a pharmaceutical composition comprising the same can be administered to a subject by any method known to a person skilled in the art. These methods include, but are not limited to, orally, parenterally, intravascularly, paracancerally, transmucosally, transdermally, intramuscularly, intranasally, intravenously, intradermally, subcutaneously, sublingually, intraperitoneally, intraventricularly, intracranially, intravaginally, by inhalation, rectally, or intratumorally. These methods include any means in which the compound or the pharmaceutical composition comprising the same can be delivered to tissue (e.g., needle or catheter). Alternatively, a topical administration may be desired for application to dermal, ocular, or mucosal surfaces. Another method of administration is via aspiration or aerosol formulation. The compound or the pharmaceutical composition may be administered topically to body surfaces and are thus formulated in a form suitable for topical administration. Suitable topical formulations include gels, ointments, creams, lotions, drops and the like. For topical administrations, the compositions are prepared and applied as solutions, suspensions, or emulsions in a physiologically acceptable diluent with or without a pharmaceutical carrier.

In preparing the compositions in oral dosage form, any of the usual pharmaceutical media may be employed. Thus, for liquid oral preparations, such as, suspensions, elixirs, and solutions, suitable carriers and additives include water, glycols, oils, alcohols, flavoring agents, preservatives, coloring agents, and the like. For solid oral preparations such as, powders, capsules, and tablets, suitable carriers and additives include starches, sugars, diluents, granulating agents, lubricants, binders, disintegrating agents, and the like. For parenteral formulations, the carrier will usually comprise sterile water, though other ingredients may be included, such as ingredients that aid solubility or for preservation. Injectable solutions may also be prepared in which case appropriate stabilizing agents may be employed. In some applications, it may be advantageous to utilize the active agent in a “vectorized” form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

Methods of treatment using formulations suitable for oral administration may be presented as discrete units such as capsules, cachets, tablets, or lozenges, each containing a predetermined amount of the active ingredient. Optionally, a suspension in an aqueous liquor or a non-aqueous liquid may be employed, such as a syrup, an elixir, an emulsion, or a draught.

A tablet may be made by compression or molding, or wet granulation, optionally with one or more accessory ingredients. Compressed tablets may be prepared by compressing in a suitable machine, with the active compound being in a free-flowing form such as a powder or granules which optionally is mixed with, for example, a binder, disintegrant, lubricant, inert diluent, surface active agent, or discharging agent. Molded tablets comprised of a mixture of the powdered active compound with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration may comprise a sterile aqueous preparation of the active compound, which, in some embodiments, is isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery. Administration may for example be intravenous, intra-arterial, intrathecal, intramuscular, subcutaneous, intramuscular, intra-abdominal (e.g., intraperitoneal), etc., and may be carried out by infusion pumps (external or implantable) or any other suitable means appropriate to the desired administration modality.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

For administration to mammals, and particularly humans, it is expected that the physician will determine the actual dosage and duration of treatment, which will be most suitable for an individual and can vary with the age, weight, genetics and/or response of the particular individual. In some embodiments, the subject is a mammal (e.g., a human or non-human mammal). In some embodiments, the subject is a human.

The methods of the invention comprise administration of a compound of the invention, or a pharmaceutically acceptable salt thereof, at a therapeutically effective amount. The therapeutically effective amount may include various dosages.

A dosage unit of the compounds used in the present invention may comprise a single compound or mixtures thereof with additional therapeutic agents. A “dose” or “dosage unit” or “unit dosage” of a compound of the invention as measured in milligrams refers to the milligrams of the compound of the invention present in a composition, regardless of the form of the composition.

In some embodiments, a dosage unit can be prepared for oral dosage forms, such as tablets, capsules, pills, powders, liquid suspensions, and granules.

In some embodiments, a compound of the invention is administered at a dosage of 1-3000 mg per day. In some embodiments, a compound of the invention as described herein is administered at a dosage of 1-1000 mg per day. In some embodiments, a compound of the invention as described herein is administered at a dosage of 1-500 mg per day. In some embodiments, a compound of the invention as described herein is administered at a dosage of 10-500 mg per day. In some embodiments, a compound of the invention as described herein is administered at a dosage of 25-500 mg per day. In some embodiments, a compound of the invention as described herein is administered at a dosage of 50-500 mg per day. In some embodiments, a compound of the invention as described herein is administered at a dosage of 5-250 mg per day. In some embodiments, a compound of the invention as described herein is administered at a dosage of 10-250 mg per day. In some embodiments, a compound of the invention as described herein is administered at a dosage of 20-250 mg per day. In some embodiments, a compound of the invention as described herein is administered at a dosage of 25-250 mg per day. In some embodiments, a compound of the invention as described herein is administered at a dosage of 25-200 mg per day. In some embodiments, a compound of the invention as described herein is administered at a dosage of 25-150 mg per day. In some embodiments, a compound of the invention as described herein is administered at a dosage of 25-125 mg per day. In some embodiments, a compound of the invention as described herein is administered at a dosage of 25-100 mg per day.

In other embodiments, a compound of the invention is administered at a dose of 1-10 mg per day, 3-26 mg per day, 3-60 mg per day, 3-16 mg per day, 3-30 mg per day, 10-26 mg per day, 10-100 mg per day, 15-60 mg per day, 15-100 mg per day, 25-100 mg per day, 50-100 mg per day, 50-200 mg per day, 100-200 mg per day, 100-250 mg per day, 125-300 mg per day, 20-50 mg per day, 5-50 mg per day, 200-500 mg per day, 125-500 mg per day, 500-1000 mg per day, 200-1000 mg per day, 1000-2000 mg per day, 1000-3000 mg per day, 125-3000 mg per day, 2000-3000 mg per day, 300-1500 mg per day or 100-1000 mg per day.

The methods may comprise administering a compound at various dosages. For example, the compound may be administered per day at a dosage of 3 mg, 10 mg, 30 mg, 40 mg, 50 mg, 80 mg, 100 mg, 120 mg, 125 mg, 200 mg, 250 mg, 300 mg, 450 mg, 500 mg, 600 mg, 900 mg, 1000 mg, 1500 mg, 2000 mg, 2500 mg or 3000 mg.

Alternatively, the compound may be administered at a dosage of 0.1 mg/kg/day. The compound may be administered at a dosage between 0.2 to 30 mg/kg/day, or 0.2 mg/kg/day, 0.3 mg/kg/day, 1 mg/kg/day, 3 mg/kg/day, 5 mg/kg/day, 10 mg/kg/day, 20 mg/kg/day, 30 mg/kg/day, 50 mg/kg/day or 100 mg/kg/day.

In some embodiments, the compound of the invention is prepared for once daily administration. In another embodiment, the compound of the invention is prepared for more than once daily administration, for example, twice daily, three times daily, four times daily, etc. In some embodiments, the compound or the pharmaceutical composition of the invention is administered in the form of a capsule, a tablet, or a liquid suspension. In other embodiments, the compound or the pharmaceutical composition of the invention is administered in an oral dosage unit form.

The methods of treatment of the present invention can additionally include administering to the subject one or more additional therapeutic agents for a combination therapy. Such additional therapeutic agents may be administered, by a route and in an amount commonly used therefore, simultaneously or sequentially with a compound or composition of the present invention.

The term “combination therapy” means the administration of two or more therapeutic agents to treat a cancer described in the present invention. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the disorders described herein.

As used herein, the singular forms “a”, “an” and “the” include plural referents unless the content clearly dictates otherwise. The terms “subject” and “patient” are used interchangeably herein when referencing, for example, a mammalian subject. In some embodiments, the subject in the method of the invention is a human patient.

The term “treatment” or “treating” as used herein refers to the administering of a therapeutically effective amount of the compound of the invention to ameliorate undesired symptoms associated with the cancer, to prevent the manifestation of such symptoms before they occur, to slow down the progression of the cancer, slow down the deterioration of symptoms, to slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

It is to be understood that the phraseology or terminology employed herein, and not otherwise defined, is for the purpose of description only and not of limitation. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. Further, reference to values stated in ranges includes each and every value within that range. Certain features of the disclosed compositions and methods which are described herein in the context of separate aspects may also be provided in combination in a single aspect. Alternatively, various features of the disclosed compositions and methods that are, for brevity, described in the context of a single aspect, may also be provided separately or in any subcombination.

### EXAMPLES

### Example 1: A Cell Viability Assay for a Compound of the Invention Shows Specificity for a Tumor Cell Line

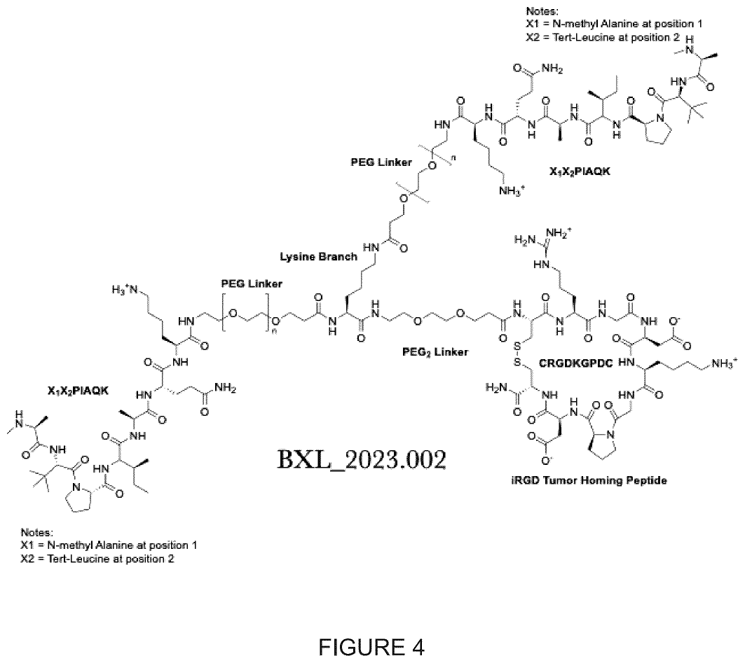

In this example, a cell viability assay was performed using the compound BXL_2023.002 (depicted in FIG. 4, and where n=8) for the pancreatic cancer cell line ASPC-1 (with the HEK-293 cell line as a control).

The cell viability assay was performed according to standard methods. Briefly, both ASPC-1 and HEK293 cells were cultured under standard conditions until ˜80% confluent, and similar passage numbers were maintained for both cell lines to minimize variability.

The cells were detached and seeded into 96-well plates at a density ensuring sub-confluence throughout the experiment. The cells were then allowed to attach overnight. The cells were treated with varying concentrations of the compound the next day and incubated. Untreated cells were included as controls.

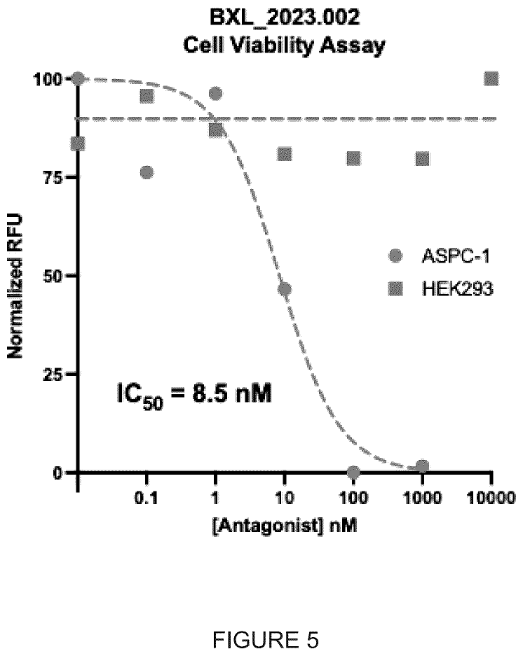

After treatment with the compound for the specified time, the cells were detached, and the cell suspension was mixed with 0.4% trypan blue solution in a 1:1 ratio. The mix was loaded onto a disposable counting slide. The cells were counted with an automatic counter, noting viable and non-viable cells. Cell viability was calculated using the formula: Cell viability (%)=(Number of viable cells/Total cells (viable+non-viable))×100. FIG. 5 is a plot of cell viability as a function of BXL_2023.002 concentration, which shows the specificity of BXL_2023.002 for pancreatic cancer cell line ASPC-1 (circles). Cell viability for the control HEK-293 cell line is also shown (squares) for comparison.

While certain features of the invention have been illustrated and described herein with some degree of particularity, it is understood that this description has been given only by way of example and that numerous changes in the details of construction, fabrication, and use, including the combination and arrangement of parts may be made without departing from the spirit and scope of the invention, and that many modifications, substitutions, changes, and equivalents will now occur to those of ordinary skill in the art. It is, therefore, to be understood that the appended claims are intended to cover all such modifications and changes as fall within the true spirit of the invention.

1. A multifunctional therapeutic peptide conjugate comprising: a tumor homing peptide, at least one protein binding moiety and a branched reactive amino acid, wherein the tumor homing peptide is conjugated to a first linker, the at least one protein binding moiety is conjugated to a second linker and the first and second linkers are conjugated to the branched reactive amino acid, wherein the at least one protein binding moiety is an inhibitor of apoptosis (IAP).

2. The peptide conjugate compound according to claim 1, wherein the tumor homing peptide comprises an active cellular targeting moiety (A) having a peptide sequence containing an RGD peptide motif consisting of the three amino acids Arg-Gly-Asp or an NGR motif consisting of the three amino acids Asn-Gly-Arg.

3. The peptide conjugate compound according to claim 1, wherein the tumor homing peptide comprises an active cellular targeting moiety (A) having a cyclic peptide sequence containing a cyclic iRGD peptide motif selected from the group consisting of Cys-Arg-Gly-Asp-Lys-Gly-Pro-Asp-Cys (SEQ ID NO: 1), Cys-Arg-Gly-Asp-Arg-Gly-Pro-Asp-Cys (SEQ ID NO: 2), Cys-Arg-Gly-Asp-Lys-Gly-Pro-Glu-Cys (SEQ ID NO: 3), and Cys-Arg-Gly-Asp-Arg-Gly-Pro-Glu-Cys (SEQ ID NO: 4).

4-6. (canceled)

7. The peptide conjugate compound according to claim 3, wherein the cyclic peptide sequence containing the cyclic iRGD peptide motif is cyclized via a disulfide bond between cysteine at residue position 1 and cysteine at residue position 9.

8. (canceled)

9. The peptide conjugate compound according to claim 1, wherein the tumor homing peptide comprises an active cellular targeting moiety (A) having a cyclic peptide sequence containing an NGR motif consisting of the five amino acids Cys-Asn-Gly-Arg-Cys (SEQ ID NO: 5).

10. The peptide conjugate compound according to claim 9, wherein the cyclic peptide sequence containing the NGR motif is cyclized with a disulfide bond between cysteine at residue position 1 and cysteine at residue position 5.

11. (canceled)

12. The peptide conjugate compound according to claim 1, wherein tumor homing peptide comprises a cyclic peptide sequence consisting of the nine amino acid sequence Cys-Gly-Asn-Lys-Arg-Thr-Arg-Gly-Cys (SEQ ID NO: 6).

13. The peptide conjugate compound according to claim 12, wherein the cyclic peptide is cyclized with a disulfide bond between cysteine at residue position 1 and cysteine at residue position 9.

14. (canceled)

15. The peptide conjugate compound according to claim 1, wherein the tumor homing peptide has a peptide sequence consisting of the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 7).

16. (canceled)

17. The peptide conjugate compound, according to claim 1, wherein the tumor homing peptide has a peptide sequence consisting of the amino acids Cys-Arg-Glu-Lys-Ala (SEQ ID NO: 8).

18. (canceled)

19. The peptide conjugate compound according to claim 1, wherein the tumor homing peptide has a peptide sequence consisting of the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 9).

20. (canceled)

21. The peptide conjugate compound of claim 1, wherein the branched reactive amino acid is a lysine, a cysteine, an azido-lysine or an azido-ornithine.

22. The peptide conjugate compound of claim 1, wherein the at least one protein binding moiety consists of a seven amino acid sequence from the N-terminus of the apoptosis mediator Smac/DIABLO protein, selected from the group consisting of AVPIAQK (SEQ ID NO: 10), [AIB]VPIAQK (SEQ ID NO: 11), and [AIB]VPI[AIB]QK (SEQ ID NO: 12), wherein [AIB] is 2-Aminoisobutyric acid.

23-24. (canceled)

25. The peptide conjugate compound of claim 1, comprising two protein binding moieties, wherein each protein binding moiety consists of a seven amino acid sequence X1X2PIAQK (SEQ ID NO: 13) and X1 is N-methyl alanine (MeA) and X2 is tert-Leucine (Tle), from the N-terminus of the apoptosis mediator Smac/DIABLO protein.

26. The peptide conjugate compound of claim 1, comprising two protein binding moieties, wherein one of the two protein binding moiety consists of a seven amino acid sequence X1X2PFAQK (SEQ ID NO: 14), wherein X1 is N-methyl alanine (MeA) and X2 is tert-Leucine (Tle) and the second of the two protein binding moiety consists of a seven amino acid sequence X1X2PIAQK (SEQ ID NO: 13), from the N-terminus of the apoptosis mediator Smac/DIABLO protein.

27. The peptide conjugate compound of claim 1 comprising two identical protein binding moieties, wherein each of the two identical protein binding moieties consists of a seven amino acid sequence from the N-terminus of the apoptosis mediator Smac/DIABLO protein selected from the group consisting of AVPIAQK (SEQ ID NO: 10), [AIB]VPIAQK (SEQ ID NO: 11), and [AIB]VPI[AIB]QK (SEQ ID NO: 12), wherein [AIB] is 2-Aminoisobutyric acid.

28-29. (canceled)

30. The peptide conjugate compound of claim 1 comprising three identical protein binding moieties, wherein each of the three identical protein binding moieties consists of a seven amino acid sequence from the N-terminus of the apoptosis mediator Smac/DIABLO protein selected from the group consisting of AVPIAQK (SEQ ID NO: 10), [AIB]VPIAQK (SEQ ID NO: 11), and [AIB]VPI[AIB]QK (SEQ ID NO: 12), wherein [AIB] is 2-Aminoisobutyric acid.

31-32. (canceled)

33. The peptide conjugate compound of claim 1, wherein each of the first linker and the second linker is an identical linker or wherein each of the first linker and the second linker is a different linker.

34. The peptide conjugate compound of claim 1, wherein the first linker and the second linker is polyethylene glycol (PEG).

35. The peptide conjugate compound of claim 1, wherein the first linker and the second linker is an identical or different polyethylene glycol (PEG) selected from the group consisting of amino-PEG2, amino-PEG3, amino-PEG4, amino-PEG6, amino-PEG8, amino-PEG10, amino-PEG12 and amino-PEG20.

36-37. (canceled)

38. A method for treating cancer comprising administering a therapeutically effective amount of the multifunctional therapeutic peptide conjugate according to claim 1.

39-72. (canceled)


SEQUENCE LISTING (14 sequences)

SEQ ID NO: 1 - protein, 9 aa, synthetic construct
  REGION 1..9: note = Description of sequence: active cellular targeting moiety
CRGDKGPDC

SEQ ID NO: 2 - protein, 9 aa, synthetic construct
  REGION 1..9: note = Description of sequence: active cellular targeting moiety
CRGDRGPDC

SEQ ID NO: 3 - protein, 9 aa, synthetic construct
  REGION 1..9: note = Description of sequence: active cellular targeting moiety
CRGDKGPEC

SEQ ID NO: 4 - protein, 9 aa, synthetic construct
  REGION 1..9: note = Description of sequence: active cellular targeting moiety
CRGDRGPEC

SEQ ID NO: 5 - protein, 5 aa, synthetic construct
  REGION 1..5: note = Description of sequence: tumor homing peptide
CNGRC

SEQ ID NO: 6 - protein, 9 aa, synthetic construct
  REGION 1..9: note = Description of sequence: tumor homing peptide
CGNKRTRGC

SEQ ID NO: 7 - protein, 29 aa, synthetic construct
  REGION 1..29: note = Description of sequence: tumor homing peptide
KDEPQRRSARLSAKPAPPKPEPKKAPAKK

SEQ ID NO: 8 - protein, 5 aa, synthetic construct
  REGION 1..5: note = Description of sequence: tumor homing peptide
CREKA

SEQ ID NO: 9 - protein, 21 aa, synthetic construct
  REGION 1..21: note = Description of sequence: tumor homing peptide
KETWWETWWTEWSQPKKKRKV

SEQ ID NO: 10 - protein, 7 aa, synthetic construct
  REGION 1..7: note = Description of sequence: N-terminus of apoptosis mediator Smac/Diablo protein, a human mitochondrial protein
AVPIAQK

SEQ ID NO: 11 - protein, 7 aa, synthetic construct
  REGION 1..7: note = Description of sequence: modified N-terminus of apoptosis mediator Smac/Diablo protein
  MOD_RES 1: note = AIB
XVPIAQK

SEQ ID NO: 12 - protein, 7 aa, synthetic construct
  REGION 1..7: note = Description of sequence: modified N-terminus of apoptosis mediator Smac/Diablo protein
  MOD_RES 1: note = AIB
  MOD_RES 5: note = AIB
XVPIXQK

SEQ ID NO: 13 - protein, 7 aa, synthetic construct
  REGION 1..7: note = Description of sequence: modified apoptosis mediator Smac/Diablo protein
  SITE 1: note = N-methyl-alanine
  SITE 2: note = tert-Leucine
ALPIAQK

SEQ ID NO: 14 - protein, 7 aa, synthetic construct
  REGION 1..7: note = Description of sequence: modified apoptosis mediator Smac/Diablo protein
  SITE 1: note = N-methyl-alanine
  SITE 2: note = tert-Leucine
ALPFAQK